(19) United States
(12) Patent Application Publication
(10) Pub. No.: US 2022/0273684 A1
(43) Pub. Date: Sep. 1, 2022

(54) Systems and Methods to Identify Pancreatic Ductal Adenocarcinoma and Uses Thereof

(71) Applicant: The Board of Trustees of the Leland Stanford Junior University, Stanford, CA (US)

(72) Inventors: Laura D. Attardi, Stanford, CA (US); Christina Curtis, Stanford, CA (US); Brittany M. Flowers, Stanford, CA (US); Hang Xu, Stanford, CA (US); Jose A. Seoane Fernandez, L'Hospitalet de Llobregat (ES)

(73) Assignee: The Board of Trustees of the Leland Stanford Junior University, Stanford, CA (US)

(21) Appl. No.: 17/653,114

(22) Filed: Mar. 1, 2022

Related U.S. Application Data
(60) Provisional application No. 63/155,156, filed on Mar. 1, 2021.

(51) Int. Cl. A61K 31/7004; C12Q 1/6886; C12Q 1/6874; G01N 33/50; C12N 5/09; A61K 31/433; A61K 31/519; A61P 35/00

(52) U.S. Cl. CPC A61K 31/7004; C12Q 1/6886; C12Q 1/6874; G01N 33/5008; C12N 5/0693; A61K 31/433; A61K 31/519; A61P 35/00; C12N 2503/02

(57) ABSTRACT

Embodiments herein describe systems and methods to identify and treat pancreatic ductal adenocarcinoma (PDAC) based on cell of origin. Various embodiments comprise assessing transcriptomic data to identify gene expression profiles for the cancer. Based on type of cancer, additional embodiments treat individuals for PDAC. Further embodiments identify possible treatments for a PDAC tumor, by screening effective treatments, including drugs.

### CROSS REFERENCE TO RELATED APPLICATIONS

This application claims priority to U.S. Provisional Application Ser. No. 63/155,156, entitled “Systems and Methods to Identify Pancreatic Ductal Adenocarcinoma and Uses Thereof,” filed Mar. 1, 2021, which is incorporated herein by reference in its entirety.

### STATEMENT REGARDING FEDERALLY SPONSORED RESEARCH OR DEVELOPMENT

This invention was made with Governmental support under Contract Nos. CA197591 and CA238296 awarded by the National Institutes of Health. The government has certain rights in the invention.

### FIELD OF THE INVENTION

The present invention relates to transcriptomic analysis, more specifically, identifying gene expression profiles that distinguish subtypes of pancreatic ductal adenocarcinoma (PDAC) and treatments associated therewith.

### BACKGROUND

Pancreatic ductal adenocarcinoma (PDAC) is a deadly cancer that is projected to be the second leading cause of cancer-related deaths in the United States by 2030. The 5-year survival rate for PDAC patients is a mere 9% and is attributable to late-stage diagnosis—when patients are rarely eligible for surgical resection—and therapeutic strategies being largely ineffective. A better understanding of how PDAC arises is vital for improving both early detection and treatment.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In one embodiment, a method for identifying pancreatic ductal adenocarcinoma (PDAC) subtype includes obtaining or having obtained transcriptomic data derived from a tumor from an individual affected with PDAC and identifying a cell origin of the tumor based on the transcriptomic data.

In additional embodiments, the method may include one or more of the following: the cell origin is selected from an acinar cell-derived tumor and a ductal cell-derived tumor; the method further includes obtaining a tumor sample from the individual affected with PDAC and performing a transcriptomic analysis on the tumor sample; the transcriptomic analysis is selected from bulk RNA analysis, spatial transcriptomic analysis, and single cell RNA sequencing; the transcriptomic analysis uses a microarray; the method further includes determining a treatment response for the tumor; determining a treatment response includes culturing a cell isolated from the tumor, providing a treatment to the cell culture, incubating the cell culture, and determining a response to the treatment based on a cell viability in the cell culture cells after incubation; the method further includes treating the individual affected with PDAC based on the response to the treatment; the method further includes treating the individual affected with PDAC based on whether the tumor is an acinar cell-derived tumor or a ductal cell-derived tumor; the tumor is a ductal cell-derived tumor; treating the individual affected with PDAC comprises targeting the glycolysis pathway; treating the individual affected with PDAC includes administering at least one of TDZD-8 and 2-DG to the individual affected with PDAC; the tumor is an acinar cell-derived tumor; treating the individual affected with PDAC comprises inhibiting AKT kinase; treating the individual affected with PDAC includes administering Capivasertib to the individual affected with PDAC.

In another embodiment, a method for determining treatment response for a pancreatic ductal adenocarcinoma (PDAC) tumor includes obtaining a culture of cancer cells, providing a treatment to the culture of cancer cells, incubating the culture of cancer cells, and determining a response to the treatment based on a cell viability in the culture of cancer cells after incubation.

In further embodiments, the method may include one or more of the following: the cells in the culture of cancer cells are derived from PDAC tumor; the cells in the culture of cancer cells are derived from an acinar cell-derived tumor or a ductal cell-derived tumor; determining a response to the treatment comprises performing a cell viability assay selected from manual cell counting, flow cytometry, or performing a methyltransferase assay; the method further includes transcriptionally profiling the culture of cancer cells to identify the cell of origin of the cells.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIGS. 1A-1G illustrate exemplary transcriptome analysis of mouse acinar and ductal cell-derived tumors in accordance with various embodiments of the invention. FIG. 1A illustrates Principal component analysis of gene expression profiles of PDACs from KT;Ptf1a^{CreER};Trp53^{fl/fl} and KT;Sox9CreER;Trp53^{fl/fl} mice. FIG. 1B illustrates a volcano plot of differentially-expressed genes in acinar and ductal cell-derived tumors. A horizontal dashed line indicates an adjusted p-value of 0.05. Vertical dashed lines indicate an absolute log₂ fold change of 1.0. Genes are color-coded based on adjusted p-value and absolute log₂ fold change cut-offs. FIG. 1C illustrates representative histological images of acinar cell-derived (n=5) or ductal cell-derived (n=5) PDACs analyzed by H&E staining and immunohistochemistry for CLDN18, TFF1, or MUC5AC. Alcian blue staining indicates mucinous gland structures within tumors. Scale Bar=100 μm. FIG. 1D illustrates a volcano plot of normal pancreatic acinar cell genes highlighted in yellow and normal pancreatic ductal genes highlighted in blue. Genes are plotted based on their expression levels in acinar and ductal cell-derived tumors. A horizontal dashed line indicates an adjusted p-value of 0.05. Vertical dashed lines indicate an absolute log₂ fold change of 1.0. FIG. 1D illustrates the top 6 expression signatures enriched in acinar cell-derived tumors relative to ductal cell-derived tumors, as identified by Metascape analysis. FIG. 1E illustrates the top 6 expression signatures enriched in ductal cell-derived tumors relative to acinar cell-derived tumors, as identified by Metascape analysis. FIG. 1G illustrates (Left) Representative histological images of PDACs in KT;Sox9CreER;Trp53^{fl/fl} (n=5) and KT;Ptf1a^{CreER};Trp53^{fl/fl} (n=5) mice analyzed by Masson's Trichrome staining. Scale Bar=100 μm. (Right) Average percentage of collagen per tumor area+/−standard deviation. Each dot indicates a mouse. *p-value<0.05 assessed by a two-tailed Student's t-test.

FIGS. 2A-2N illustrate an exemplary comparison of acinar and ductal cell-derived tumor signatures to molecular subtypes of human PDAC in accordance with various embodiments of the invention. FIG. 2A illustrates (Top) Scheme summarizing how acinar and ductal cell-derived tumor signatures were derived. (Bottom) A heat map of the genes defining the signatures. FIG. 2B illustrates principal component (PC) analysis on a nanoString spatial transcriptomics data set. FIG. 2C illustrates a heatmap with hierarchical clustering (cluster 1 to 5). FIG. 2D illustrates a deconvolution analysis to identify cell type signatures from sets of genes in each cluster. FIG. 2F illustrates a scatter plot of acinar cell-derived tumor and ductal cell-derived tumor signature enrichment scores in primary PDAC samples (ICGC-AU, n=96) classified as squamous, pancreatic progenitor, immunogenic, or ADEX molecular subtypes. Each dot represents a tumor sample, color coded by molecular subtype. FIG. 2G illustrates a box plot of the ductal cell-derived tumor signature enrichment score in primary PDAC samples (ICGC-AU, n=96) classified as squamous, pancreatic progenitor, immunogenic, or ADEX molecular subtypes. The ductal cell-derived signature enrichment score is significantly lower in tumors classified as the pancreatic progenitor (n=30), ADEX (n=16), or immunogenic (n=25) subtypes than the squamous subtype (n=25), based on a two-tailed Student's t-test. ** p-value<0.01 ***p-value<0.001, ****p-value<0.0001. FIG. 2H illustrates a box plot of the acinar cell-derived tumor signature enrichment score in primary PDAC samples (ICGC-AU, n=96) classified as squamous, pancreatic progenitor, immunogenic, or ADEX molecular subtypes. The acinar cell-derived signature enrichment score is significantly higher in tumors classified as the pancreatic progenitor (n=30), ADEX (n=16), or immunogenic (n=25) subtype than the squamous subtype (n=25), based on a two-tailed Student's t-test. **p-value<0.01 ***p-value<0.001, ****p-value<0.0001. FIG. 2I illustrates a scatter plot of acinar cell-derived tumor and ductal cell-derived tumor signature enrichment scores in primary PDAC samples (ICGC-AU, n=96) classified as quasi-mesenchymal, classical, or exocrine-like molecular subtypes. Each dot represents a tumor sample, color coded by molecular subtype. FIG. 2J illustrates a box plot of the ductal cell-derived tumor signature enrichment score in primary PDAC samples (ICGC-AU, n=96) classified as quasi-mesenchymal, classical, or exocrine-like molecular subtypes. The ductal cell-derived signature enrichment score is significantly lower in tumors classified as the classical (n=39) or exocrine-like (n=29) subtype than the quasi-mesenchymal subtype (n=28), based on a two-tailed Student's t-test. ** p-value<0.01 ***p-value<0.001. FIG. 2K illustrates a box plot of the acinar cell-derived tumor signature enrichment score in primary PDAC samples (ICGC-AU, n=96) classified as quasi-mesenchymal, classical, or exocrine-like molecular subtypes. The acinar cell-derived signature enrichment score is significantly higher in tumors classified as the classical (n=39) or exocrine-like (n=29) subtype than the quasi-mesenchymal subtype (n=28), based on a two-tailed Student's t-test. *p-value<0.05. FIG. 2L illustrates a scatter plot acinar cell-derived tumor and ductal cell-derived tumor signature enrichment scores in primary PDAC samples (ICGC-AU, n=96) classified as basal-like or classical molecular subtypes. Each dot represents a tumor sample, color coded by molecular subtype. FIG. 2M illustrates a box plot of the ductal cell derived tumor signature enrichment score in primary PDAC samples (ICGC-AU, n=96) classified as basal-like or classical molecular subtypes. The ductal cell-derived signature enrichment score is significantly higher in tumors classified as the basal-like (n=44) than the classical (n=52) subtype, based on a two-tailed Student's t-test. ***p-value<0.001. FIG. 2N illustrates a box plot of the acinar cell derived tumor signature enrichment score in primary PDAC samples (ICGC-AU, n=96) classified as basal-like or classical molecular subtypes. The acinar cell-derived signature enrichment score is significantly lower in tumors classified as the basal-like (n=44) than the classical (n=52) subtype, based on a two-tailed Student's t-test. ****p-value<0.0001.

FIGS. 3A-3D illustrate the cell-of-origin signature is associated with survival outcomes in accordance with various embodiments of the invention. FIG. 3A illustrates Kaplan-Meier analysis of the Australian ICGC PDAC patient cohort overall survival stratified by expression of the acinar cell-derived tumor signature. Patients with high expression of the acinar cell-derived tumor signature (n=48) exhibit significantly better survival than in patients with low expression of the acinar cell-derived signature (n=47) based on the log-rank test. ****p-value<0.0001. FIG. 3B illustrates Kaplan-Meier analysis of the Australian ICGC PDAC patient cohort overall survival stratified by the ductal cell-derived tumor signature. Patients with high expression of the ductal cell-derived tumor signature (n=47) exhibit significantly worse survival than patients with low expression of the ductal cell-derived signature (n=49) based on the log-rank test. *p-value<0.05. FIG. 3C illustrates Cox proportional hazards ratios of the association between age, tumor, stage or grade, 4 subtype PDAC classification (ADEX, immunogenic, pancreatic progenitor, or squamous) or the acinar or ductal cell-derived tumor signature enrichment (where high expression is reflective of enrichment) and overall survival of the Australian ICGC patient cohort (ICGC-AU; n=96). Asterisks indicate the factors that are significantly associated with overall survival. *p-value<0.05, **p-value<0.01, ***p-value<0.001. FIG. 3D illustrates Meta-analysis of the cox proportional hazards ratios of the acinar or ductal cell-derived tumor signature enrichment (where high expression is reflective of enrichment) in the Australian ICGC (ICGC-AU), Canadian ICGC (ICGC-CA), and TCGA patient cohorts, shown individually and combined. Asterisks indicate individual cohorts where the cell-of-origin signature enrichment is significantly associated with overall survival. *p-value<0.05, ***p-value<0.001.

FIGS. 4A-4E illustrate exemplary data that acinar and ductal cell-derived pancreatic cancer cells have different sensitivities to multiple therapeutic agents in accordance with various embodiments of the invention. FIG. 4A illustrates an exemplary method to identify agents to treat specific types of tumors and/or treating an individual based on tumor signature. FIG. 4B illustrates a schematic of the experimental setup. FIG. 4C illustrates cell viability after a 24 hour incubation with 25 μM of GSK-3β inhibitor, TDZD-8. FIG. 4D illustrates cell viability after a 48 hour incubation with 1 mM of glucose analog, 2-DG. FIG. 4E illustrates cell viability after a 72 hour incubation with 100 μM of AKT kinase inhibitor, Capivasertib.

FIG. 5 illustrates an exemplary method to identify and/or treat an individual for PDAC in accordance with various embodiments of the invention.

FIGS. 6A-6G illustrate exemplary data showing pancreatic cancer can arise from adult mouse acinar cells in accordance with various embodiments of the invention. FIG. 6A illustrates a schematic for acinar cell-derived pancreatic cancer study of KT;Ptf1a^{CreER}; Trp53^{+/+}, KT;Ptf1a^{CreER};Trp53^{fl/+}, and KT;Ptf1a^{CreER};Trp53^{fl/fl} mouse cohorts. Mice were treated with tamoxifen for 3 consecutive days at 8-10 weeks of age, then aged to analyze pancreatic cancer-free survival. FIG. 6B illustrates a representative co-immunofluorescence staining for tdTomato (TOM), amylase (AMY; acinar cell marker), cytokeratin 19 (CK19; ductal cell marker), and insulin (INS; islet marker), in a Rosa26^{LSL-tdTomato/LSL-tdTomato};Ptf1a^{CreER};Trp53^{+/+} mouse pancreas 3 days after the last dose of tamoxifen (n=5). DAPI stains nuclei. Scale Bar=50 μm. FIG. 6C illustrates Kaplan-Meier analysis of pancreatic cancer-free survival of cohorts listed in FIG. 6A. Labels indicate the Trp53 status of each cohort. Pancreatic cancer-free survival in KT;Ptf1a^{CreER};Trp53^{fl/fl} mice (n=23) and KT;Ptf1aC^{CreER};Trp53^{fl/+} mice (n=6) is significantly shorter than that in KT;Ptf1a^{CreER};Trp53^{+/+} mice (n=21), based on the log-rank test. Pancreatic cancer-free survival in KT;Ptf1a^{CreER};Trp53^{fl/fl} mice (n=23) is significantly shorter than that in KT;Ptf1a^{CreER};Trp53^{+/+} mice (n=6), based on the log-rank test. ***p-value<0.001, ****p-value<0.0001. FIG. 6D illustrates a table summarizing the percentages of tumor-bearing mice in KT;Ptf1a^{CreER};Trp53^{+/+} (n=9), KT;Ptf1a^{CreER};Trp53^{fl/+} (n=6), and KT;Ptf1a^{CreER};Trp53^{fl/fl} (n=23) cohorts presenting with clinical symptoms of pancreatic cancer (ascites, bowel obstruction, and jaundice) at morbidity. FIG. 6E illustrates a table summarizing the percentages of tumor-bearing KT;Ptf1a^{CreER};Trp53^{+/+} (n=9), KT;Ptf1a^{CreER};Trp53^{fl/+} (n=6), and KT;Ptf1a^{CreER};Trp53^{fl/fl} (n=23) mice with primary tumor grade (comprising>50% of the tumor) called as moderately/well-differentiated adenocarcinoma, poorly-differentiated adenocarcinoma, or sarcomatoid carcinoma. The primary tumor grades of KT;Ptf1a^{CreER}-Trp53^{+/+} mice are significantly different from KT;Ptf1a^{CreER};Trp53^{fl/fl} mice by the Fisher's Exact Test (p-value<0.05). FIG. 6F illustrates representative histological images of lesions found in each cohort analyzed by H&E staining and immunohistochemistry for tdTomato and CK19. Alcian blue staining marks PanINs. Scale Bar=100 μm. FIG. 6G illustrates representative histological images of metastases to the liver and lungs in KT;Ptf1a^{CreER};Trp53^{fl/fl} mice analyzed by H&E staining and immunohistochemistry for tdTomato and CK19. Scale Bar=100 μm.

FIGS. 7A-7G illustrate exemplary analysis of p53 missense mutants in acinar cell-derived pancreatic cancer development in accordance with various embodiments of the invention. FIG. 7A illustrates a schematic for mutant p53 acinar cell-derived pancreatic cancer study of KT;Ptf1a^{CreER};Trp53^{fl/-}, KT;Ptf1a^{CreER};Trp53^{fl/LSL-R172H}, and KT;Ptf1a^{CreER};Trp53^{fl/LSL-R270H} mouse cohorts. Mice were treated with tamoxifen for 3 consecutive days at 8-10 weeks of age, then aged to analyze pancreatic cancer-free survival. The genotypes of tumor cells and stromal cells after Cre action are indicated. FIG. 7B illustrates Kaplan-Meier analysis of pancreatic cancer-free survival of cohorts listed in (A). Labels indicate the Trp53 status of each cohort. Pancreatic cancer-free survival of KT;Ptf1a^{CreER};Trp53^{fl/-} mice (n=23) is significantly shorter than that of KT;Ptf1a^{CreER};Trp53^{fl/LSL-R172H} mice (n=25) but similar to that of KT;Ptf1a^{CreER};Trp53^{fl/LSL-R270H} mice (n=28), based on the log-rank test. Pancreatic cancer-free survival is similar in KT;Ptf1a^{CreER};Trp53^{fl/LSL-R172H} and KT;Ptf1a^{CreER};Trp53^{fl/LSL-R270H} mice, based on the log-rank test. **p-value<0.01. Not significant=ns. FIG. 7C illustrates a table summarizing the percentages of KT;Ptf1a^{CreER};Trp53^{fl/-} (n=23), KT;Ptf1a^{CreER};Trp53^{fl/LSL-R172H} (n=25), and KT;Ptf1a^{CreER};Trp53^{fl/LSL-R270H} (n=27) mice presenting with clinical symptoms of pancreatic cancer (ascites, bowel obstruction, and jaundice) at morbidity. FIG. 7D illustrates a table summarizing the percentages of tumor-bearing KT;Ptf1a^{CreER};Trp53^{fl/-} (n=23), KT;Ptf1a^{CreER};Trp53^{fl/LSL-R172H} (n=25), and KT;Ptf1a^{CreER};Trp53^{fl/LSL-R270H} (n=27) mice with primary tumor grade (comprising >50% of the tumor) called as moderately/well-differentiated adenocarcinoma or poorly-differentiated adenocarcinoma as well as the frequencies of metastasis to the liver. All cohorts have similar primary tumor grades and metastasis frequencies, by Fisher's Exact Test. With the exception with of one mouse that developed a thymic lymphoma, necessitating early sacrifice, all of the mice in the KT;Ptf1a^{CreER};Trp53^{fl/LSL-R270H} cohort had evidence of PDAC. FIG. 7E illustrates representative histological images of adenocarcinomas found in each cohort, analyzed by H&E staining and immunohistochemistry for tdTomato and CK19. Scale Bar=100 μm. FIG. 7F illustrates representative histological image of a liver metastasis in a KT;Ptf1a^{CreER};Trp53^{fl/-} mouse analyzed by H&E staining and immunohistochemistry for tdTomato and CK19. Scale Bar=100 μm. FIG. 7G illustrates examples of p53-negative and mutant p53-positive primary pancreatic tumors in a KT;Ptf1a^{CreER}Trp53^{fl/LSL-R172H} mouse as well as p53-negative and mutant p53-positive liver metastases in KT;Ptf1a^{CreER};Trp53^{fl/LSL-R172H} and KT;Ptf1a^{CreER};Trp53^{fl/LSL-R270H} mice, respectively, analyzed by H&E staining and p53 immunohistochemistry. Scale Bar=100 μm.

FIGS. 8A-8H illustrate exemplary data showing pancreatic cancer can develop from adult mouse ductal cells in accordance with various embodiments of the invention. FIG. 8A illustrates a schematic for ductal cell-derived pancreatic cancer study of KT;Sox9CreER;Trp53^{+/+} and KT;Sox9CreER;Trp53^{fl/fl} mouse cohorts. Mice were treated with tamoxifen for 3 consecutive days at 8-10 weeks of age, then aged to analyze survival. FIG. 8B illustrates representative co-immunofluorescence staining for tdTomato (TOM) and amylase (AMY; acinar cell marker), cytokeratin 19 (CK19; ductal cell marker), and insulin (INS; islet marker), in a Rosa26^{LSL-tdTomato/LSL-tdTomato};Sox9CreER;Trp53^{+/+} mouse pancreas 3 days after the last dose of tamoxifen (n=4). DAPI stains nuclei. Scale Bar=50 μm. FIG. 8C illustrates Kaplan-Meier analysis of pancreatic cancer-free survival of cohorts listed in (A). Labels indicate the Trp53 status of each cohort. Pancreatic cancer-free survival in KT;Sox9CreER;Trp53^{fl/fl} mice (n=25) is significantly different from that of KT;Sox9CreER;Trp53^{+/+} mice (n=18), based on the log-rank test. ***p-value<0.001. FIG. 8D illustrates Kaplan-Meier analysis of overall survival of cohorts listed in (A). Labels indicate the Trp53 status of each cohort. Overall survival in KT;Sox9CreER;Trp53^{fl/fl} mice (n=25) is significantly different from that of KT;Sox9CreER;Trp53^{+/+} mice (n=18), based on the log-rank test. “***p-value<0.0001. FIG. 8E illustrates a table summarizing the percentages of pancreatic tumor-bearing KT;Sox9CreER;Trp53^{fl/fl} mice presenting with clinical symptoms of pancreatic cancer (ascites and bowel obstruction) at morbidity (n=9). FIG. 8F illustrates a table summarizing the percentages of tumor-bearing KT;Sox9CreER;Trp53^{fl/fl} mice (n=9) with primary tumor grade (comprising >50% of the tumor) of moderately/well-differentiated or poorly-differentiated adenocarcinomas. KT;Sox9CreER;Trp53^{+/+} mice (n=18) had no evidence of pancreatic tumors. FIG. 8G illustrates representative histological images of typical pancreas morphology found in each cohort, analyzed by H&E staining and immunohistochemistry for tdTomato and CK19. Scale Bar=100 μm. FIG. 8H illustrates a representative image of a peritoneum metastasis in a KT;Sox9CreER;Trp53^{fl/fl} mouse analyzed by H&E staining and immunohistochemistry for tdTomato and CK19. Scale Bar=100 μm.

FIGS. 9A-9E illustrate exemplary analysis of the precursors in PDAC development from acinar and ductal cells in accordance with various embodiments of the invention. FIG. 9A illustrates (Left) Representative histological image of pancreas in a KT;Sox9CreER;Trp53^{fl/fl} mouse at −120 days post-tamoxifen analyzed by co-immunofluorescence for AMY (acinar cell marker) and CK19 (ductal cell marker; n=6). DAPI stains nuclei. Scale Bar=200 μm. (Right) High magnification image of area within dashed box by co-immunofluorescence and H&E staining. Scale Bar=100 μm. FIG. 9B illustrates representative histological image of pancreas in a KT;Ptf1a^{CreER};Trp53^{fl/fl} mouse at −70 days post-tamoxifen analyzed by co-immunofluorescence for AMY (acinar cell marker) and CK19 (ductal cell marker; n=7). DAPI stains nuclei. Scale Bar=200 μm. (Right) High magnification image of area within dashed box by co-immunofluorescence and H&E staining. Scale Bar=100 μm. FIG. 9C illustrates a representative histological image of a tumor found in a KT;Sox9CreER;Trp53^{fl/fl} mouse analyzed by H&E staining. Scale Bar=400 μm. (Inset) High magnification of area within dashed box analyzed by immunohistochemistry for tdTomato. Black dashed curved line separates tumor from non-tumor area. Scale Bar=100 μm. FIG. 9D illustrates a representative histological image of a tumor found in a KT;Ptf1a^{CreER};Trp53^{fl/fl} mouse, analyzed by H&E staining. White dashed curved line roughly separates tumor from non-tumor area. Scale Bar=400 μm. (Inset) High magnification of area within black dashed box analyzed by immunohistochemistry for tdTomato. Alcian blue staining indicates PanINs. Scale Bar=100 μm. FIG. 9E illustrates representative phospho-ERK1/2 immunostaining of the pancreas in KT;Sox9CreER;Trp53^{fl/fl} (n=6) and KT;Ptf1a^{CreER};Trp53^{fl/fl} (n=7) mice at approximately 120 and 70 days, respectively, the midpoint of median survival. Alcian blue staining marks PanINs. Each arrowhead denotes an example of a phospho-ERK1/2 expressing duct, ADM, or PanIN. Scale Bar=50 μm.

### DETAILED DESCRIPTION

Turning now to the drawings, systems and methods to identify pancreatic ductal adenocarcinoma (PDAC) and uses thereof are provided. Many embodiments provide methods that identify subtypes of PDAC based on transcriptomic analysis, including by identifying gene expression profiles associated with specific subtypes of PDAC. Additional embodiments provide methods to identify drugs to which the different subtypes respond.

The genetics underlying PDAC development are well-described. Human PDAC genome sequencing has identified several recurrent genomic alterations in pancreatic tumors, including activating mutations in KRAS in >90% of PDACs and mutations in the TP53, CDKN2A, and SMAD4 tumor suppressor genes in 72%, 30%, and 32% of cases, respectively. (See e.g., Ying H, et al. Genetics and biology of pancreatic ductal adenocarcinoma. Genes Dev 2016; 30(4):355-85 doi 10.1101/gad.275776.115; Waters A M, Der C J. KRAS: The Critical Driver and Therapeutic Target for Pancreatic Cancer. Cold Spring Harb Perspect Med 2018; 8(9) doi 10.1101/cshperspect.a031435; and Aguirre A J, Hruban R H, Raphael B J, Canc Genome Atlas Res N. Integrated Genomic Characterization of Pancreatic Ductal Adenocarcinoma. Cancer Cell 2017; 32(2):185-+doi 10.1016fj.ccell.2017.07.007; the disclosures of which are hereby incorporated by reference in their entireties.) Analysis of human pancreas samples through DNA and immunohistochemical analyses has led to a proposed progression model for PDAC, with oncogenic KRAS mutations serving as the initiating event and specific tumor suppressor gene mutations occurring at defined stages thereafter to promote cancer progression. (See e.g., Hruban R H, Goggins M, Parsons J, Kern S E. Progression model for pancreatic cancer. Clin Cancer Res 2000; 6(8):2969-72; the disclosure of which is hereby incorporated by reference in its entirety.) In this model, cancers arise when oncogenic KRAS promotes the formation of preinvasive Pancreatic Intraepithelial Neoplasias (PanINs) that progress through increasingly more dysplastic stages, culminating in frank carcinomas and metastasis. Studies in genetically engineered mouse models in which oncogenic KRAS is expressed and tumor suppressor genes are inactivated in the pancreas have been instrumental for demonstrating the importance of these combined mutations in driving PDAC development. (See e.g., Hingorani S R, et al. Preinvasive and invasive ductal pancreatic cancer and its early detection in the mouse. Cancer Cell 2003; 4(6):437-50 doi 10.1016/s1535-6108(03)00309-x; Hingorani S R, et al. Trp53(R172H) and KraS(G12D) cooperate to promote chromosomal instability and widely metastatic pancreatic ductal adenocarcinoma in mice. Cancer Cell 2005; 7(5):469-83 doi 10.1016fj.ccr.2005.04.023; Aguirre A J, et al. Activated Kras and Ink4a/Arf deficiency cooperate to produce metastatic pancreatic ductal adenocarcinoma. Genes Dev 2003; 17(24):3112-26 doi 10.1101/gad.1158703; Bardeesy N, et al. Smad4 is dispensable for normal pancreas development yet critical in progression and tumor biology of pancreas cancer. Genes Dev 2006; 20(22):3130-46 doi 10.1101/gad.1478706; and Bardeesy N, et al. Both p16(Ink4a) and the p19(Arf)-p53 pathway constrain progression of pancreatic adenocarcinoma in the mouse. Proc Natl Acad Sci USA 2006; 103(15):5947-52 doi 10.1073/pnas.0601273103; the disclosures of which are hereby incorporated by reference in their entireties.) These models faithfully reproduce the entire progression of human PDAC, from neoplastic lesions to metastatic cancer.

As a complementary approach to genomic analyses, transcriptomic analyses have been utilized to define molecular subtypes of human PDAC, with the goal of identifying distinct classes that could guide clinical decision-making. Three seminal studies established a framework for PDAC classification. Depending on the methodology, transcriptomics analysis of resected PDAC samples from untreated patients revealed between 2 and 4 distinct molecular subtypes. (See e.g., Bailey P, et al. Genomic analyses identify molecular subtypes of pancreatic cancer. Nature 2016; 531(7592):47-52 doi 10.1038/nature16965; Moffitt R A, et al. Virtual microdissection identifies distinct tumor- and stroma-specific subtypes of pancreatic ductal adenocarcinoma. Nature Genet 2015; 47(10):1168-78 doi 10.1038/ng.3398; Collisson E A, et al. Subtypes of pancreatic ductal adenocarcinoma and their differing responses to therapy. Nat Med 2011; 17(4):500-3 doi 10.1038/nm.2344; the disclosures of which are hereby incorporated by reference in their entireties.) While differences in sample cellularity and technology used for transcriptomic analysis may underlie the differences in specific subtypes defined by each study, there is a consensus from these initial studies as well as subsequent ones that two broad pancreatic cancer subtypes exist: the classical and the basal-like subtypes, with the latter being associated with poorer prognosis. (See e.g., Collisson E A, et al. Subtypes of pancreatic ductal adenocarcinoma and their differing responses to therapy. Nat Med 2011; 17(4):500-U140 doi 10.1038/nm.2344; Moffitt R A, et al. Virtual microdissection identifies distinct tumor- and stroma-specific subtypes of pancreatic ductal adenocarcinoma. Nature Genet 2015; 47(10):1168-+doi 10.1038/ng.3398; Bailey P, et al. Genomic analyses identify molecular subtypes of pancreatic cancer. Nature 2016; 531(7592):47-+doi 10.1038/nature16965; Collisson E A, et al. Molecular subtypes of pancreatic cancer. Nat Rev Gastroenterol Hepatol 2019; 16(4):207-20 doi 10.1038/s41575-019-0109-y; Aguirre A J, Hruban R H, Raphael B J, Canc Genome Atlas Res N. Integrated Genomic Characterization of Pancreatic Ductal Adenocarcinoma. Cancer Cell 2017; 32(2):185-203 doi 10.1016/j.ccell.2017.07.007; Hayashi A, et al. A unifying paradigm for transcriptional heterogeneity and squamous features in pancreatic ductal adenocarcinoma. Nature Cancer 2020; 1(1):59-74 doi 10.1038/s43018-019-0010-1; Maurer C, et al. Experimental microdissection enables functional harmonisation of pancreatic cancer subtypes. Gut 2019; 68(6):1034-43 doi 10.1136/gutjnl-2018-317706; Puleo F, et al. Stratification of Pancreatic Ductal Adenocarcinomas Based on Tumor and Microenvironment Features. Gastroenterology 2018; 155(6):1999-2013 e3 doi 10.1053fj.gastro.2018.08.033; and Chan-Seng-Yue M, et al. Transcription phenotypes of pancreatic cancer are driven by genomic events during tumor evolution. Nat Genet 2020; 52(2):231-40 doi 10.1038/s41588-019-0566-9; the disclosures of which are hereby incorporated by reference in their entireties.) The cellular and molecular factors that drive each molecular subtype, however, remain unknown. Unequivocally establishing the factors critical for dictating the subtypes will refine our understanding of the underlying biology of these classes and ultimately improve risk stratification and therapeutic development. Such an understanding, however, requires tractable genetic experiments in model systems to allow interrogation of such factors as specific gene mutations and cell type-of-origin on the development of different subtypes.

Correlative analyses have suggested particular genetic associations with specific subtypes, such as mutations in the TP53 gene, which encodes the p53 transcription factor, with the basal-like subtype. TP53 commonly sustains missense mutations in the DNA binding domain that not only induce loss of wild-type p53 function, but may also promote gain-of-function properties through the production of a mutant p53 protein. (See e.g., Kim M P, Lozano G. Mutant p53 partners in crime. Cell Death Differ 2018; 25(1):161-8 doi 10.1038/cdd.2017.185; the disclosure of which is hereby incorporated by reference in its entirety.) Missense mutations comprise two major classes: contact mutations that alter residues required by p53 to contact DNA and structural mutations that affect the three-dimensional structure of the protein. (See e.g., Mello S S, Attardi L D. Not all p53 gain-of-function mutants are created equal. Cell Death Differ 2013; 20(7):855-7 doi 10.1038/cdd.2013.53; the disclosure of which is hereby incorporated by reference in its entirety.) The most frequently observed TP53 mutations in PDAC are at codons 175 and 273 (corresponding to mouse codons 172 and 270, respectively), which represent structural and contact residue mutations. (See e.g., Bouaoun L, et al. TP53 Variations in Human Cancers: New Lessons from the IARC TP53 Database and Genomics Data. Hum Mutat 2016; 37(9):865-76 doi 10.1002/humu.23035; the disclosure of which is hereby incorporated by reference in its entirety.) While the majority of mouse PDAC studies have relied on combined oncogenic KRAS and p53R172H expression to drive metastatic PDAC, oncogenic KRAS and Trp53 deletion also promote metastatic PDAC. (See e.g., Mello S S, et al. A p53 Super-tumor Suppressor Reveals a Tumor Suppressive p53-Ptpn14-Yap Axis in Pancreatic Cancer. Cancer Cell 2017; 32(4):460-73 e6 doi 10.1016/j.ccell.2017.09.007; the disclosure of which is hereby incorporated by reference in its entirety.) How Trp53 deletion and different classes of p53 point mutants impact the development of different subtypes of PDAC, however, remains unexplored.

It also remains unclear how cell-of-origin influences pancreatic cancer subtype. Acinar and ductal cells are the major epithelial cell types in the exocrine pancreas, but which of these serves as the cell-of-origin in PDAC has been debated. Early studies in mouse PDAC models failed to reveal the cell-of-origin because they relied on strains in which Cre recombinase is expressed in multipotent pancreas progenitor cells during embryogenesis. While PanINs resemble ducts and were originally thought to arise from normal ductal cells, more recent evidence using tamoxifen-regulatable Cre to induce genetic mutations in adult mice has suggested that acinar cells can give rise to PanINs and PDAC through a cellular reprogramming process termed Acinar-to-Ductal Metaplasia (ADM). (See e.g., Habbe N, et al. Spontaneous induction of murine pancreatic intraepithelial neoplasia (mPanIN) by acinar cell targeting of oncogenic Kras in adult mice. Proc Natl Acad Sci USA 2008; 105(48):18913-8 doi 10.1073/pnas.0810097105; Lee A Y L, et al. Cell of origin affects tumour development and phenotype in pancreatic ductal adenocarcinoma. Gut 2019; 68(3):487-98 doi 10.1136/gutjnl-2017-314426; Friedlander S Y G, et al. Context-Dependent Transformation of Adult Pancreatic Cells by Oncogenic K-Ras. Cancer Cell 2009; 16(5):379-89 doi 10.1016fj.ccr.2009.09.027; Guerra C, et al. Chronic pancreatitis is essential for induction of pancreatic ductal adenocarcinoma by k-Ras Oncogenes in adult mice. Cancer Cell 2007; 11(3):291-302 doi 10.1016fj.ccr.2007.01.012; Bailey J M, et al. p53 mutations cooperate with oncogenic Kras to promote adenocarcinoma from pancreatic ductal cells. Oncogene 2016; 35(32):4282-8 doi 10.1038/onc.2015.441; Kopp J L, et al. Identification of Sox9-Dependent Acinar-to-Ductal Reprogramming as the Principal Mechanism for Initiation of Pancreatic Ductal Adenocarcinoma. Cancer Cell 2012; 22(6):737-50 doi 10.1016/j.ccr.2012.10.025; Guerra C, et al. Pancreatitis-Induced Inflammation Contributes to Pancreatic Cancer by Inhibiting Oncogene-Induced Senescence. Cancer Cell 2011; 19(6):728-39 doi 10.1016fj.ccr.2011.05.011; Ferreira R M M, et al. Duct- and Acinar-Derived Pancreatic Ductal Adenocarcinomas Show Distinct Tumor Progression and Marker Expression. Cell Reports 2017; 21(4):966-78 doi 10.1016/j.celrep.2017.09.093; and Ji B, et al. Ras activity levels control the development of pancreatic diseases. Gastroenterology 2009; 137(3):1072-82, 82 e1-6 doi 10.1053fj.gastro.2009.05.052; the disclosures of which are hereby incorporated by reference in their entireties.) Some evidence from adult mouse PDAC models also supports the notion that PDAC can arise from ductal cells, in the context of oncogenic KRAS expression and homozygous Trp53 or Fbw7 deletion. Thus, human PDAC likely originates from pancreatic acinar or ductal cells.

Many embodiments described herein identify factors that influence the development of different transcriptional subtypes of PDAC using a comprehensive panel of tractable genetically engineered mouse models. Various embodiments utilize transcriptomic analysis of tumors arising from acinar and ductal cells to form profiles. Further embodiments use these profiles to illuminate a connection between cell-of-origin and human PDAC subtype. Such embodiments reveal that different cells-of-origin can influence the ultimate molecular subtype of PDAC, providing critical new insight into the basis for intertumoral PDAC heterogeneity.

### Acinar and Ductal Cell-Derived Tumors are Distinct

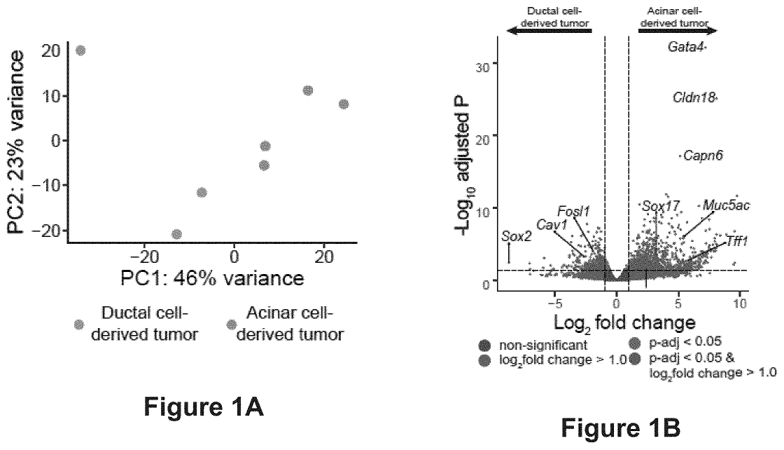

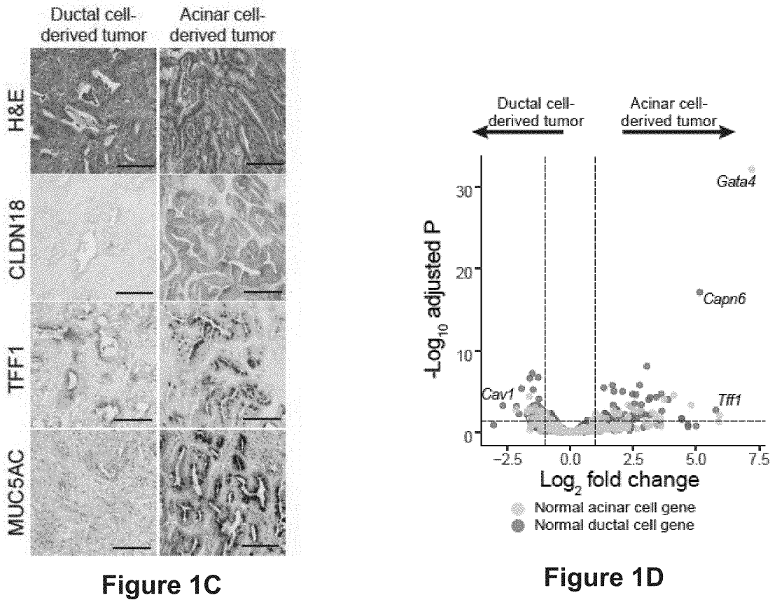

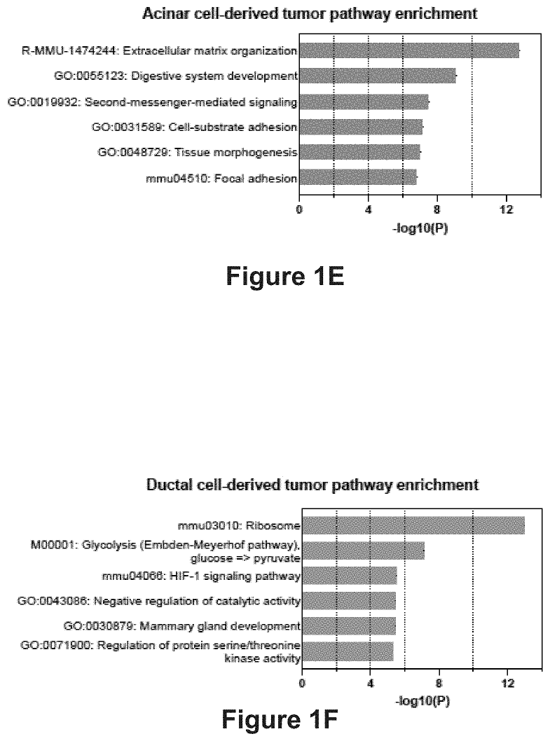

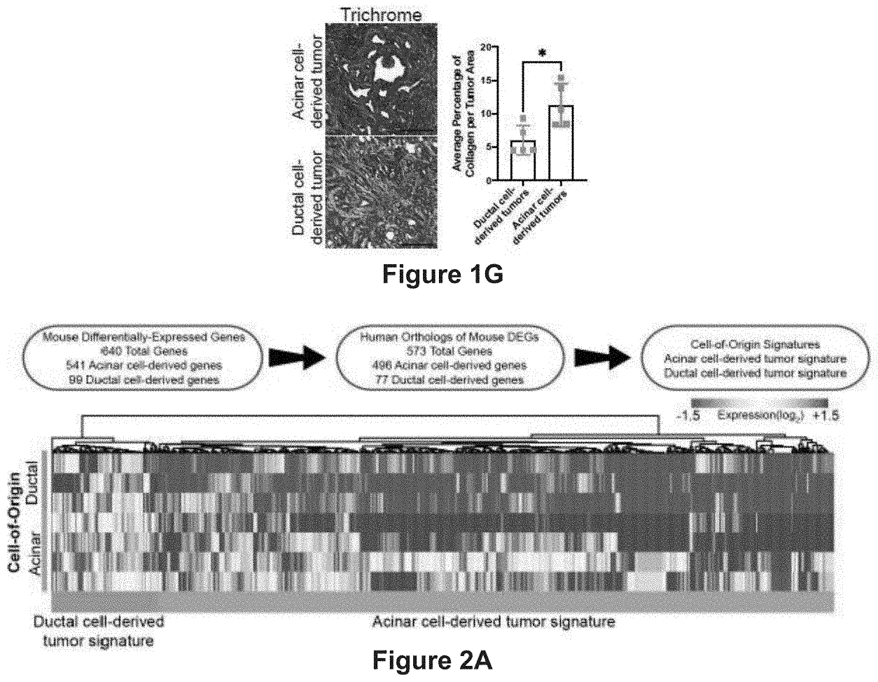

Many embodiments identify PDAC subtype based on cell-of-origin profiling. In various embodiments, the cell-of-origin is identifiable based on transcriptional signatures. FIG. 1A illustrates exemplary data showing transcriptomic data that acinar cell derived and ductal cell derived tumors cluster distinctly, suggestive of different molecular profiles.

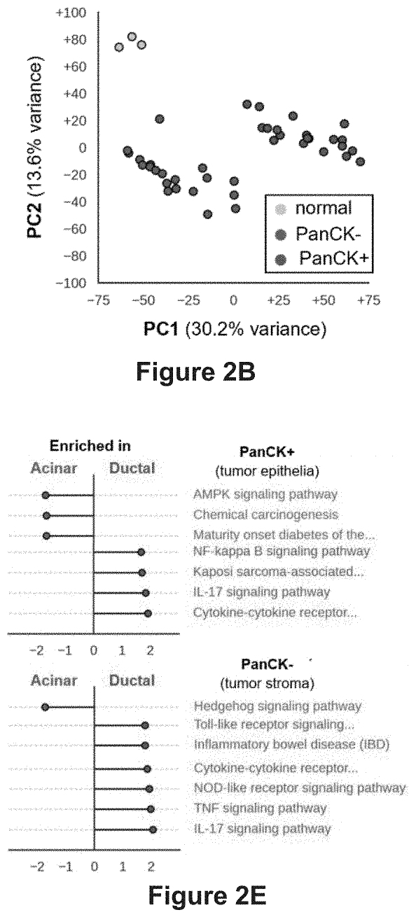

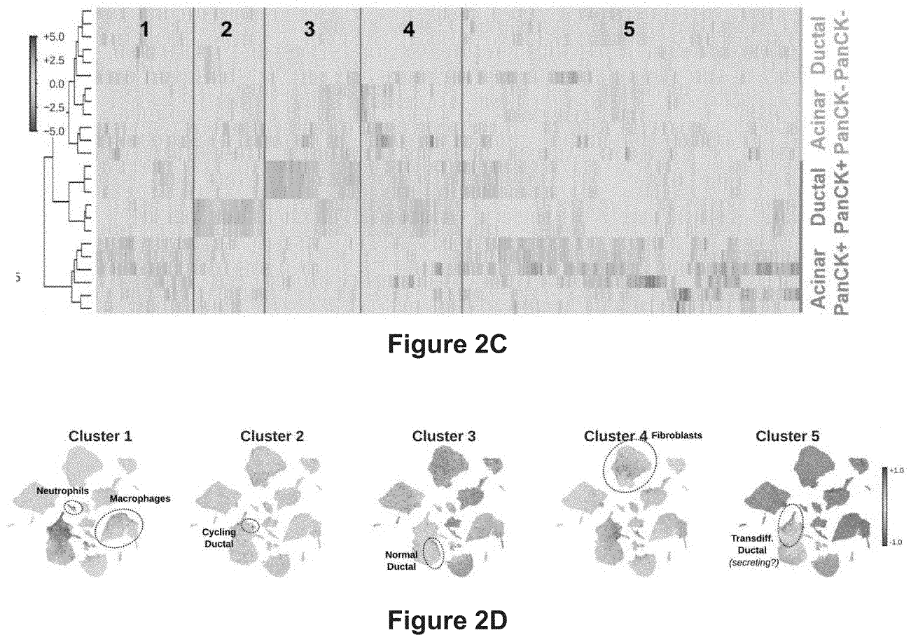

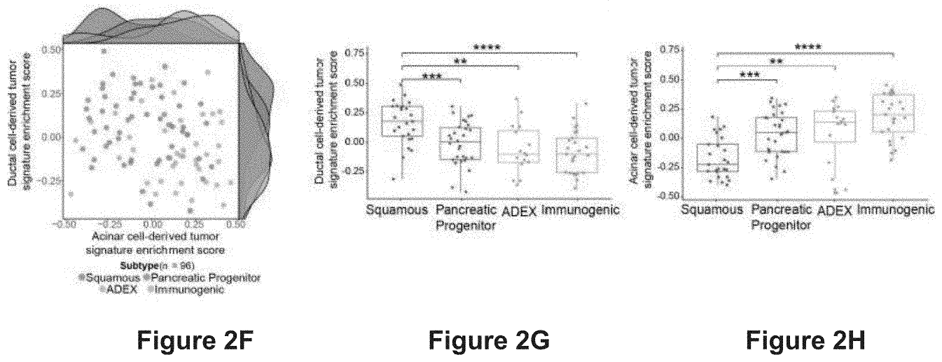

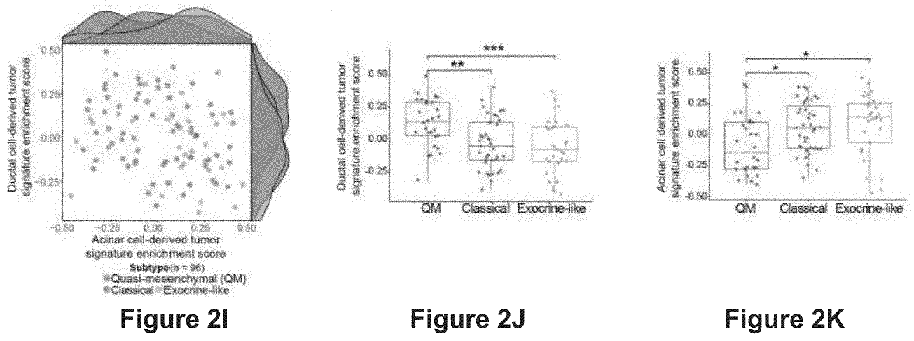

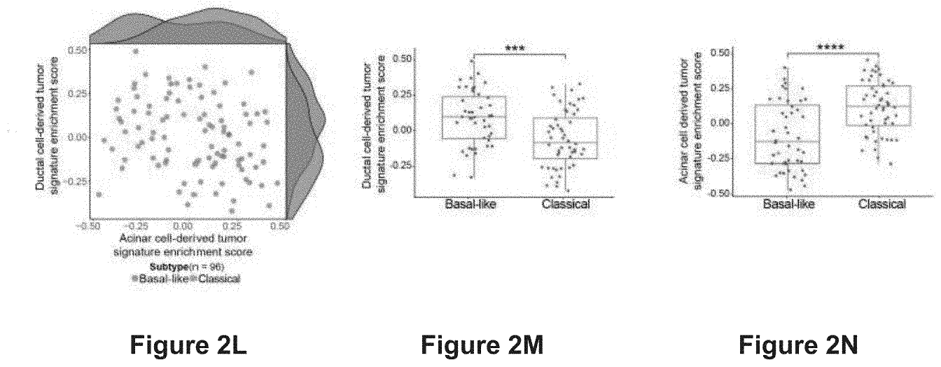

FIG. 1B shows similar exemplary data generating from RNA sequencing, where 1,075 genes are more highly expressed in acinar cell derived tumors than in ductal cell derived tumors and that 417 genes are more highly expressed in ductal cell derived tumors than in acinar cell derived tumors (log₂ fold change>1.0, p-adjusted value<0.05). FIG. 1C illustrates additional exemplary data immunohistochemical analysis showing differential protein expression between ductal cell derived tumors and acinar cell derived tumors. Turning to FIGS. 1E-1F, functional annotations of genes enriched in each of acinar cell derived tumors (FIG. 1E) and ductal cell derived tumors (FIG. 1F) are enriched). The functional annotations of FIGS. 1E-1F show that ductal-derived tumors were characterized by upregulation of pathways such as glycolysis and HIF1 pathways, while acinar-derived tumors were characterized by extracellular and tissue reorganization. Understanding the various pathways that are differentially expressed can indicate possible pathways to target, or not target, to treat tumors derived from a particular cell type. In FIG. 2A, gene enrichment profiles of human genes for acinar and ductal cell derived tumors are illustrated. Table 1 further lists genes associated with acinar and ductal cell derived tumors.

Turning to FIGS. 2B-2E, exemplary spatial transcriptomic is illustrated. Specifically, FIG. 2B illustrates independent clustering between tumor epithelia (PanCK+) and tumor stroma (PanCK−) in principal component analysis. FIG. 2C illustrates exemplary unsupervised hierarchical clustering to identify subsets of genes showing coordinated expression changes between PanCK+(tumor epithelia) and PanCK− (tumor stroma) regions of PDACs of acinar- and ductal-cell of origin. This clustering reveals five clusters, where clusters 1 and 3 show genes that are more specifically upregulated in ductal cell-derived samples, in both PanCK+ and PanCK−, respectively, while cluster 4 and 5 show those specifically upregulated in acinar cell-derived PanCK− and PanCK+, respectively. FIG. 2D illustrates exemplary deconvolution analysis showing that cluster 1 signatures are enriched in signatures and pathways representing neutrophils and macrophages, indicating that ductal-derived tumors have higher infiltrating innate immune cells in the PanCK− microenvironment than the acinar-derived tumors. The signature of cluster 1 is associated with poorer prognosis by mediating therapeutic resistance through immunosuppression. In cluster 4, acinar-derived tumors show upregulated fibroblast signatures, which often are involved in various processes of extracellular matrix organization and tissue morphogenesis. Additionally, FIG. 2E illustrates exemplary data of gene set enrichment that identifies distinct pathways that are specifically induced in tumor cells of different origins, which are also reflected by their microenvironment.

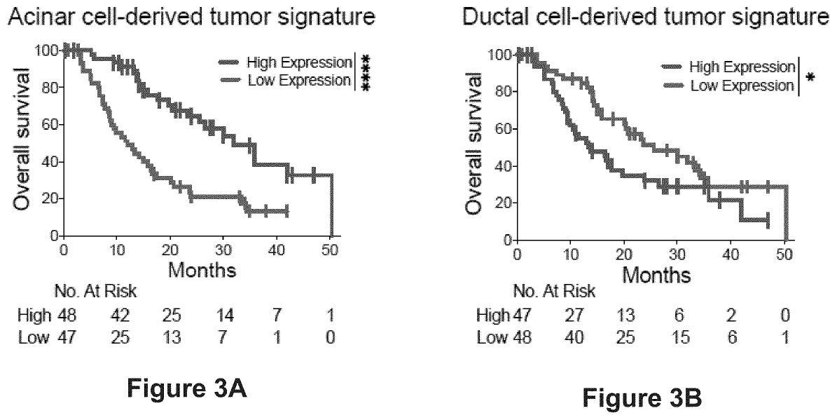

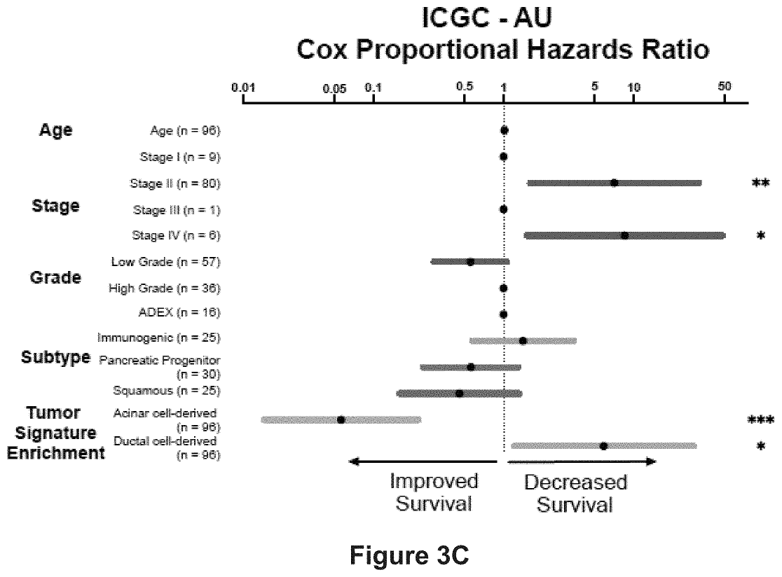

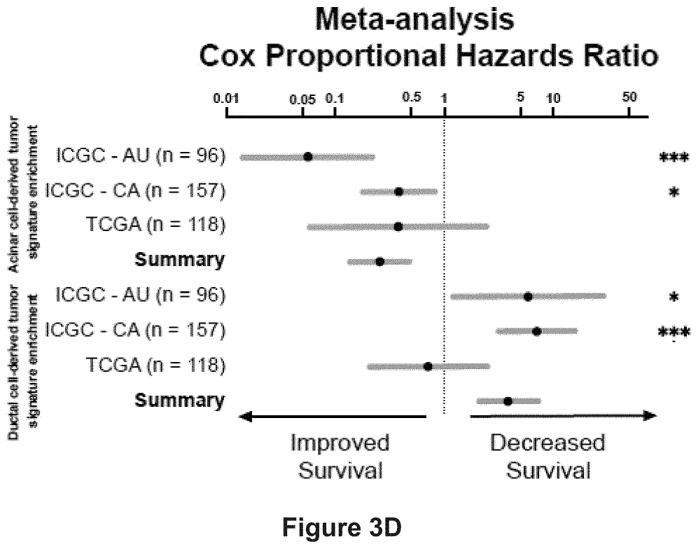

Turning to FIGS. 3A-3B, acinar cell-derived and ductal cell-derived signatures exhibit different survival outcomes. Specifically, FIG. 3A illustrates exemplary data showing that patients whose tumors showed low expression of the acinar cell-derived signature had significantly worse overall survival than those with high expression of the acinar signature. In contrast, FIG. 3B illustrates exemplary data showing that patients whose tumors showed high expression of the ductal cell-derived signature had significantly worse overall survival than those with low expression of the ductal signature.

### Identifying Effective Treatments

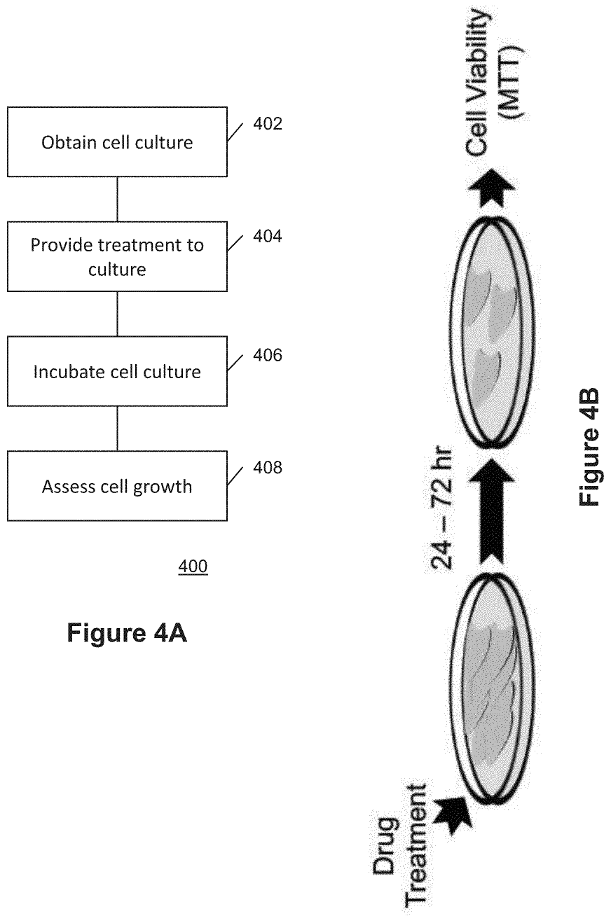

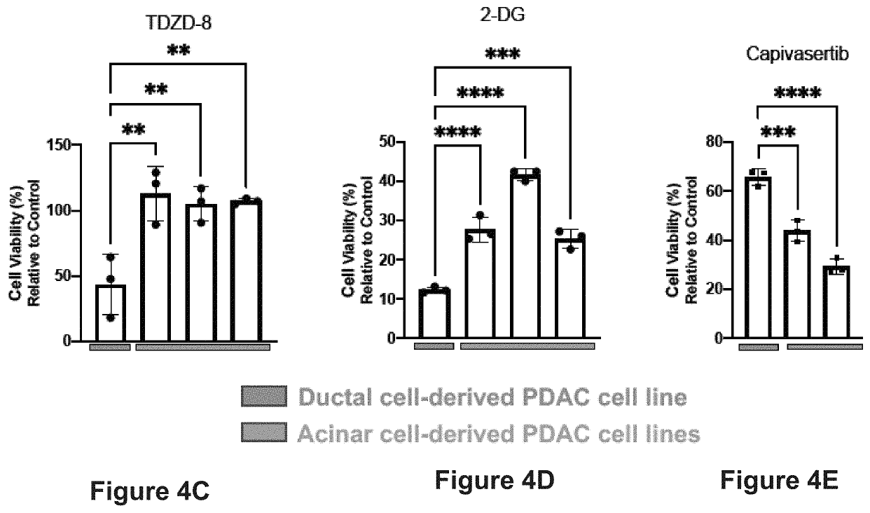

Turning to FIGS. 4A-4E, various embodiments are capable of identifying agents to treat specific types of tumors and/or treating an individual based on tumor signature. Specifically, FIG. 4A illustrates a method 400 to test one or more treatments against tumor-derived cells. At 402, many embodiments obtain one or more cultures of cells. In various embodiments, the derived tumor cells are obtained as an admixture of cells, while some embodiments obtain cells as a single type (e.g., healthy, acinar cell-derived, or ductal cell-derived). In admixed cells, cells can be cultured as various monocultures, specific for individual cells. Further embodiments profile the cells to classify the cells for their origin (e.g., healthy, acinar cell-derived, or ductal cell-derived), as a confirmation of cell type or to discover cell type. Cell cultures can be plated onto a solid or semi-solid media (e.g., agar) or liquid culture. Cell cultures can be grown in 2D (e.g., plate) or a 3D matrix. Many embodiments alter culturing methods and conditions to optimize or adapt for a particular cell culture, which are appreciated to one of skill in the art.

Many embodiments provide a treatment (e.g., control, drug, biologic, immunologic, radiation, etc.) to the one or more cultures of cells at 404. Various embodiments provide the treatment into an existing culture, apply the treatment as a beam, or apply the drug by transferring cells to a new culture media containing the treatment. FIG. 4B graphically illustrates a cell culture being treated with a drug at 452.

Returning to FIG. 4A, additional embodiments incubate the treated culture for a period of time at 406. Such incubation can be at any combination of time, temperature, and other conditions to allow sufficient effect caused by the treatment. In various embodiments, the incubation progresses for 24-72 hours and at temperatures between 20° C.-37° C. Additional embodiments alter atmospheric or gaseous content within a culture, such that certain embodiments incubate the culture at atmospheric concentrations of oxygen (e.g., approximately 20%), while some embodiments reduce oxygen levels below atmospheric concentrations of oxygen (e.g., 5%, 7%, 10%, etc.) Under non-control treatments, cultures are expected to have reduced cellular growth and/or cellular death. FIG. 4B illustrates an incubation with reduced cellular count (e.g., from cell death) after a period of incubation with a drug treatment at 454.

Turning back to FIG. 4A, after treatment, many embodiments assess cellular growth at 408. Cellular growth can be assessed manually or automatedly. Manual counts can assess the number of cells in a portion or all of the culture, while automated methods include computer-based cell counters, flow cytometers, and/or any other automated method for assessing cellular health or count within a culture. Certain embodiments utilize a methyltransferase (MTT) assay, such as illustrated in FIG. 4B, to determine cellular viability.

Based on the foregoing method 400, various embodiments are capable of identifying specific treatments, classes of treatments, and assessing the efficacy of such treatments. Additionally, it should be noted that various embodiments may omit, duplicate, and/or alter the order (including performing simultaneously) of the features of method 400, for specific applications or uses. For example, certain embodiments may assess cellular growth 408 before providing a treatment 404, as to form a baseline count or assessment of the cells in culture. One of skill in the art will understand how other features may be altered in accordance with embodiments.

FIGS. 4C-4E illustrate exemplary data of cell viability assays in accordance with many embodiments. FIGS. 4C-4D illustrate exemplary data showing that acinar cell-derived PDAC cell lines have increased viability in the presence of TDZD-8, a GSK-3p inhibitor (FIG. 4C), and 2-DG, a glucose analog (FIG. 4D), relative to ductal cell-derived PDAC lines. As will be described below, TDZD-8 and 2-DG target the glycolysis pathway, which is enriched in ductal cell-derived tumor signatures, indicating another possible route to identify possible treatment candidates. In contrast, ductal cell-derived PDAC cell lines have increased viability in the presence of Capivasertib, an AKT kinase inhibitor (FIG. 4E), relative to acinar cell-derived PDAC lines. As such, FIGS. 4C-4E illustrate that acinar cell-derived PDAC cell lines and ductal cell-derived PDAC cell lines are susceptible to different treatments. Additionally, FIGS. 4C-4E suggest that targeting glycolysis pathways may more efficacious for ductal cell-derived tumors, while inhibiting AKT kinase may be more beneficial for acinar cell-derived tumors.

### Methods of Treatment

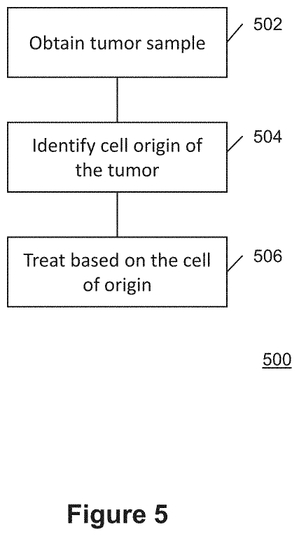

Many embodiments are directed to methods to identify and/or treat an individual for PDAC. FIG. 5 illustrates an exemplary method 500 to identify and/or treat an individual for PDAC. At 502, many embodiments obtain a tumor sample from an individual affected with PDAC (e.g., suffering from PDAC, diagnosed with PDAC, suspected of having PDAC, etc. Certain embodiments obtain the tumor sample from a biopsy, including resection, punch, blood spot, aspirate, and/or any other method to obtain such a tissue sample. Certain embodiments obtain a sample from a third party (e.g., medical practitioner), while others obtain the sample directly from an individual via one of the aforementioned methodologies. The individual with PDAC can be someone with known or suspected PDAC.

Various embodiments identify a cell origin of the tumor at 504. Certain embodiments perform a transcriptomic analysis on the tumor sample to identify a gene expression profile for the tumor sample. In certain embodiments, transcriptomic analysis is performed via bulk RNA analysis (e.g., entire tumor), while other embodiments perform spatial transcriptomic analysis to assess a specific cell type within the tumor sample. Further embodiments perform single cell RNA sequencing to assess the transcriptome of a cell or multiple cells within the tumor. Some embodiments perform a cytological assessment of the tumor sample, such as through one or more markers, antigens, stain, other cellular identifier, cell morphology, tissue morphology, and combinations thereof. Certain embodiments utilize a microarray to assess gene expression in the tumor. The cell origin identified in 504 includes acinar cell-derived tumors and/or ductal cell derived tumors, where each cell origin possesses a signature, such as those described herein.

At 506, many embodiments treat an individual based on the cell of origin (e.g., acinar cell derived or ductal cell derived). In some embodiments, the glycolysis pathway is targeted in ductal cell-derived tumors, while AKT kinase can be inhibited in acinar cell-derived tumors. Various embodiments provide TDZD-8 and/or 2-DG to target the glycolysis pathway, while other embodiments use Capivasertib as an AKT kinase inhibitor. It should be noted that TDZD-8, 2-DG, and Capivasertib are merely examples of possible therapeutics, and one of skill in the art would be aware of additional therapeutics and therapeutically effective doses that can be used in accordance with various embodiments.

It should be noted that method 500 is merely exemplary, and one of skill in the art would understand modifications and/or alterations to the method which are within the scope of various embodiments. Such modifications and/or alterations can include omitting features, repeating features, changing the order of features, and/or performing certain features simultaneously. For example, some embodiments may repeat a treatment step multiple times, in case of a more effective treatment plan or schedule. Additionally, certain embodiments may obtain transcriptomic data that has been generated by another individual.

### EXEMPLARY EMBODIMENTS

Although the following embodiments provide details on certain embodiments of the inventions, it should be understood that these are only exemplary in nature, and are not intended to limit the scope of the invention.

### Example 1: Oncogenic KRAS Activation can Induce Metastatic PDAC from Adult Pancreatic Acinar Cells

Background: To deconstruct the genetic requirements for pancreatic cancer development from adult mouse pancreatic acinar cells, a system was established to activate oncogenic Kras^{G12D} and modulate Trp53 in mouse pancreatic acinar cells through the use a tamoxifen-inducible knock-in Ptf1a^{CreER} allele.

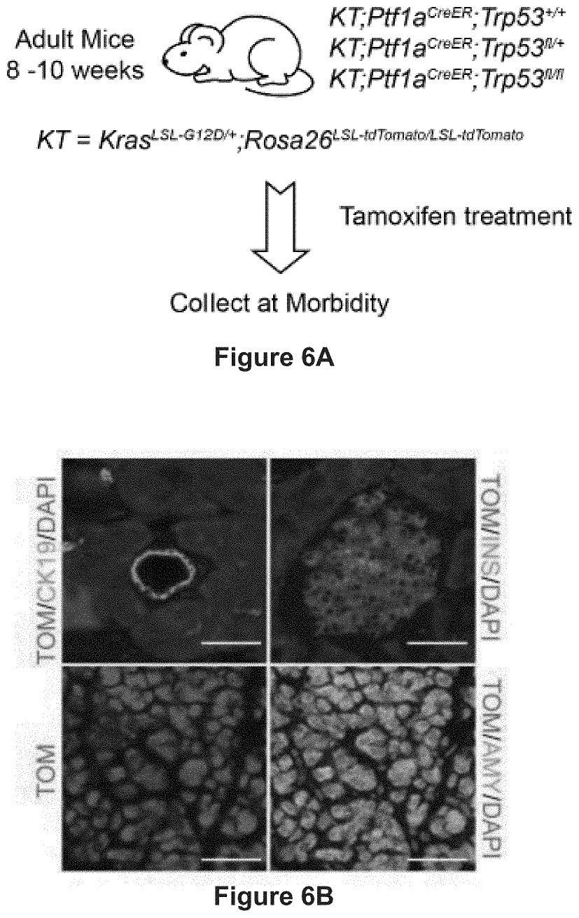

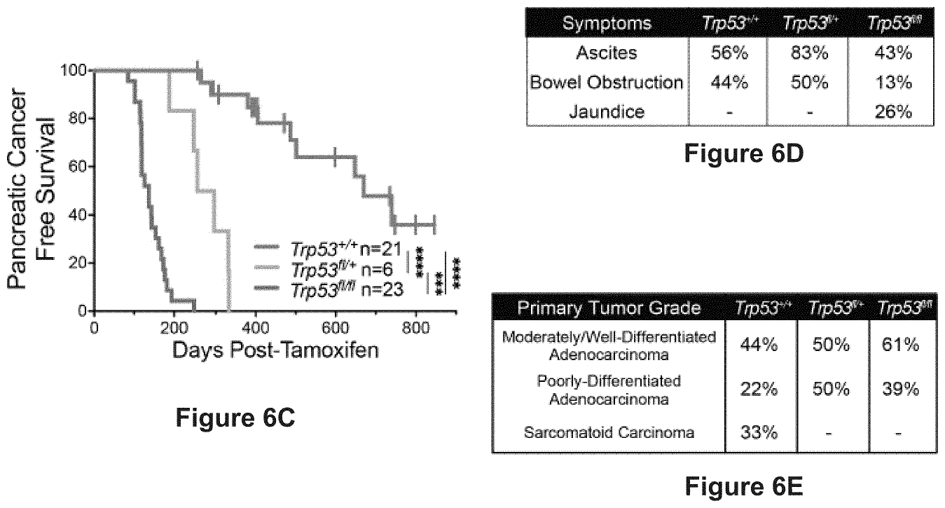

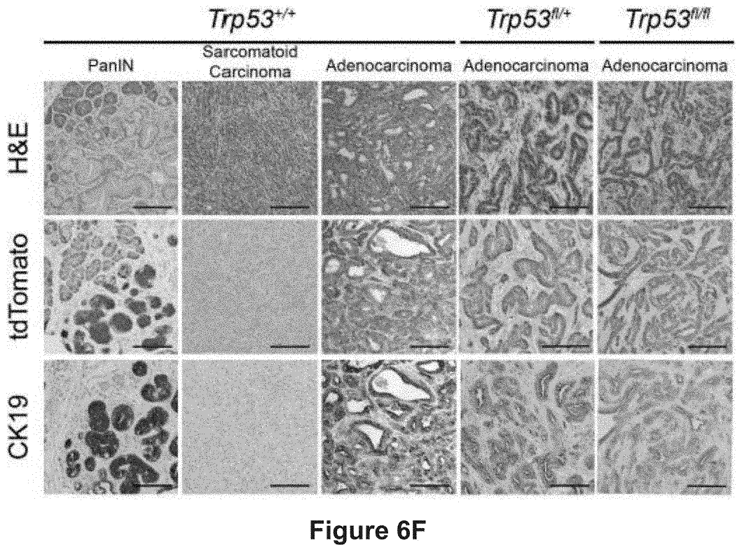

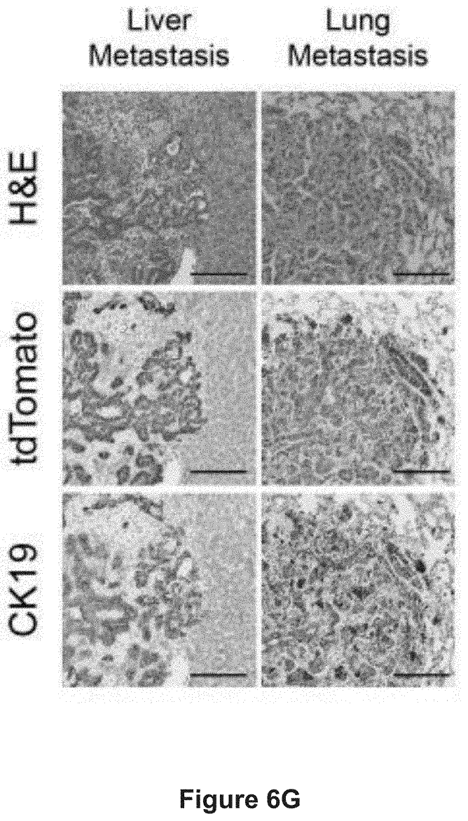

Methods: Importantly, to induce genetic alterations in cells of the fully developed adult pancreas, mice were aged to adulthood (8-10 weeks) before tamoxifen treatment (FIG. 6A). Tamoxifen treatment induced efficient genomic recombination in the majority of acinar cells, but not other pancreatic cell types, as indicated by the selective expression of the tdTomato reporter allele in amylase-positive acinar cells (FIG. 6B). To initially assess tumor development in the presence and absence of p53, cohorts of Kras^{LSL-G12D/+};Rosa26^{LSL-tdTomato/LSL-tdTomato};Ptf1a^{CreER};Trp53^{+/+} (Kras^{LSL-G12D/+};Rosa26^{LSL/tdTomato/LSL-tdTomato} heretofore denoted as KT), KT; Ptf1a^{CreER};Trp53^{fl/+}, and KT;Ptf1a^{CreER};Trp53^{fl/fl} mice were generated, tamoxifen treated, aged, and examined for pancreatic cancer-free survival upon morbidity.

Results: Kaplan-Meier analysis revealed first that loss of p53 in KT;Ptf1a^{CreER};Trp53^{fl/fl} mice led to rapid, fully penetrant development of PDAC, with a median pancreatic cancer-free and overall survival of 137 days after tamoxifen treatment (FIG. 6C). As expected, KT;Ptf1a^{CreER};Trp53^{fl/+} mice with heterozygous expression of wild-type Trp53 displayed a longer tumor latency with a median pancreatic cancer-free survival of 276.5 days after tamoxifen treatment. Interestingly, KT;Ptf1a^{CreER};Trp53^{+/+} mice also succumbed to pancreatic cancer, albeit with a significantly longer latency of 670 days. Approximately half of this latter cohort developed pancreatic cancer, with the rest displaying widespread premalignant PanIN lesions at morbidity. Of note, male KT;Ptf1a^{CreER};Trp53^{+/+} mice displayed significantly shorter pancreatic cancer-free and overall survival than female mice, in contrast to KT;Ptf1a^{CreER};Trp53^{fl/fl} mice, where survival was similar between sexes. At morbidity, some mice in all cohorts exhibited common human clinical symptoms of PDAC such as ascites, jaundice, and bowel obstruction (FIG. 6D).

Histopathological analysis provided additional insight into PDAC development. A primary and secondary histological grade were assigned for each tumor ( ) and found that KT;Ptf1a^{CreER};Trp53^{fl/+} and KT;Ptf1a^{CreER};Trp53^{fl/fl} mice consistently developed moderately/well-differentiated or poorly-differentiated adenocarcinomas characterized by Cytokeratin 19 (CK19) positivity (FIGS. 6E-6F). Of tumor-bearing KT;Ptf1a^{CreER};Trp53^{+/+} mice, 44% developed moderately/well-differentiated adenocarcinomas, 22% developed poorly-differentiated adenocarcinomas, and 33% developed sarcomatoid carcinomas, a statistically significant shift in spectrum relative to KT;Ptf1a^{CreER};Trp53^{fl/fl} mice. These tumors were either CK19-positive adenocarcinomas or sarcomatoid carcinomas with a notable positivity for vimentin and absence of CK19 immunostaining (FIG. 6F). The majority of tumors in mice of all genotypes had tumor-adjacent PanIN lesions, as indicated by alcian blue positivity (FIG. 6F). Interestingly, one KT;Ptf1a^{CreER};Trp53^{+/+} mouse had evidence of a rare intraductal papillary mucinous neoplasm (IPMN; FIG. S1F). Lineage tracing using the tdTomato allele verified that all PanINs, IPMNs, tumors, and metastases originated from Ptf1a-expressing cells, suggesting an acinar cell origin (FIGS. 6F-6G). In addition, metastatic lesions were observed in all cohorts of mice at both the gross and histological levels. Liver metastases were the most common, with rare peritoneum, diaphragm, and lung metastases observed upon dissection in some animals (FIG. 6G). Quantification of liver metastases by histological analysis revealed similar rates of metastasis, with 33% and 32% of tumors metastasizing in p53-expressing and p53-deficient mice, respectively.

Conclusion: Collectively, these findings demonstrate that oncogenic KRAS expression can drive pancreatic cancer development from adult mouse acinar cells. Although PDAC can develop in mice initially expressing wild-type p53, the latency of tumor development is significantly reduced with targeted p53 inactivation, underscoring the importance of p53 as a barrier to pancreatic cancer development in adults.

### Example 2: Expression of p53 Missense Mutants does not Reduce the Latency of Acinar Cell-Derived PDAC Development

Background: To assess how the expression of mutant p53, given its potential gain-of-function effects, compares to complete p53 deficiency in adult acinar cell-derived pancreatic cancer development, mouse cohorts were generated with oncogenic KRAS^{G12D} expression and expression of the p53 structural mutant p53^{R172H}(KT;Ptf1a^{CreER};Trp53^{fl/LSL-R172H}), the p53 contact mutant p53^{R270H} (KT;Ptf1a^{CreER};Trp53^{fl/LSL-R270H}) or p53 deficiency (KT;Ptf1a^{CreER};Trp53^{fl/-}).

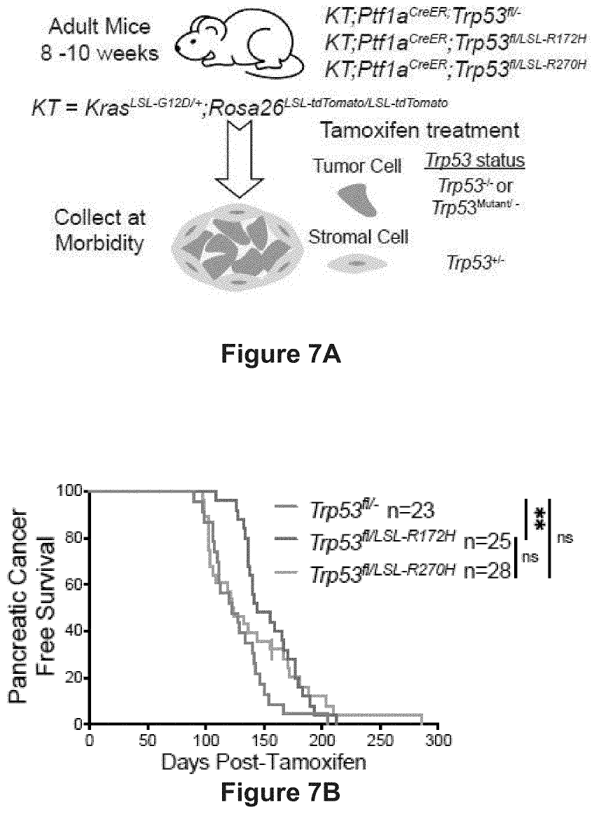

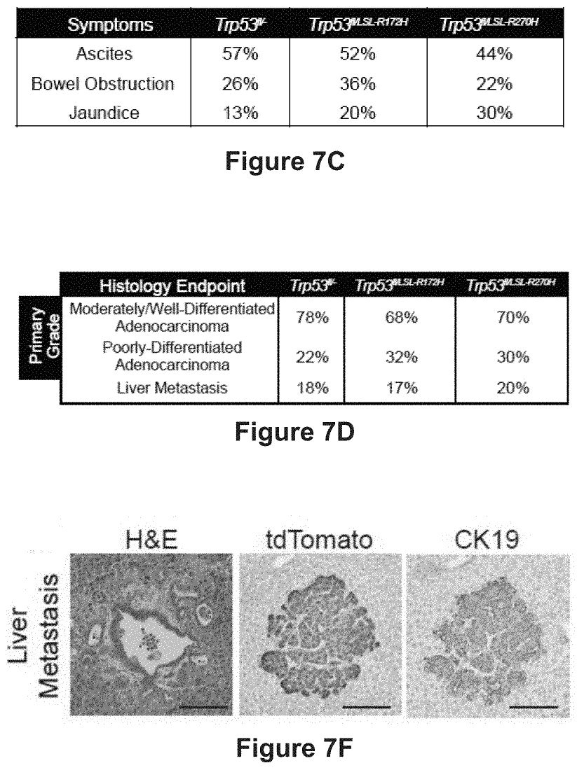

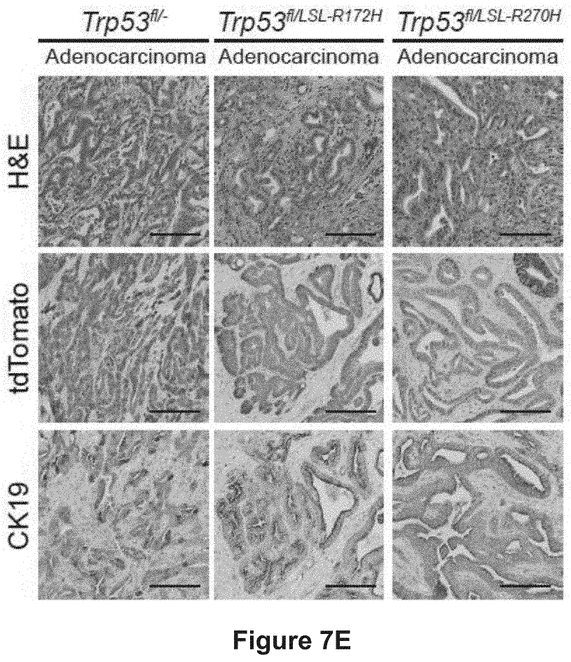

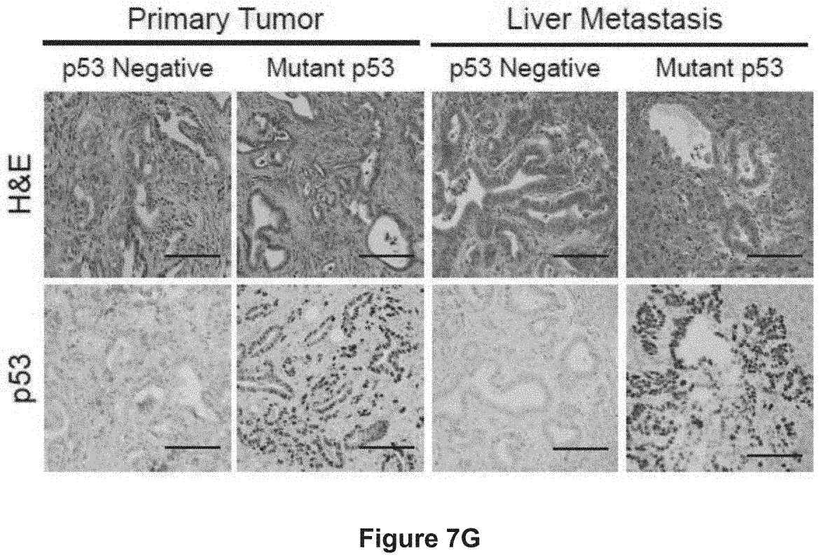

Methods: Hemizygous cohorts were generated in which expression of a Trp53 point mutant allele and a Trp53 null allele in the pancreas allows a clear assessment of potential gain-of-function activity rather than dominant-negative activity observed when the mutants are combined with a wild-type Trp53 allele. Importantly, by using a combination of a Trp53 foxed allele and conditional Lox-stop-lox activatable alleles for the Trp53 mutants (which are Trp53 null until Cre is active), mice were generated with a uniform Trp53 heterozygous genetic background in stromal tissues, for a controlled comparison of pancreatic cancer-free survival across all genotypes (FIG. 7A).

Results: Analysis of these cohorts revealed that KT;Ptf1a^{CreER};Trp53^{fl/-} mice deficient for p53 rapidly developed cancer with a median pancreatic cancer-free survival of 123 days after tamoxifen treatment (FIG. 7B). This latency is similar to that seen in KT;Ptf1a^{CreER};Trp53^{fl/fl} mice, suggesting that the Trp53 heterozygous stroma does not significantly change tumor latency. Furthermore, KT;Ptf1a^{CreER};Trp53^{fl/LSL-R270H} mice expressing the contact mutant p53^{R270H} had a similar pancreatic cancer-free survival of 123.5 days to KT;Ptf1a^{CreER};Trp53^{fl/-} mice (FIG. 7B), indicating that there is no significant gain-of-function effect driven by p53^{R270H} expression in terms of tumor latency. Interestingly, analysis of the p53^{R172H} structural mutant revealed a slightly enhanced pancreatic cancer-free survival of 144 days in the KT;Ptf1a^{CreER};Trp53^{fl/LSL-R172H} mice relative to KT;Ptf1a^{CreER};Trp53^{fl/-} mice with Trp53 deficiency, again indicating no clear gain-of-function effects with this mutant in terms of tumor latency (FIG. 7B). As with KT;Ptf1a^{CreER};Trp53^{fl/fl} mice, there was no significant difference in pancreatic cancer-free survival of female and male mice in each cohort. Some mice in all cohorts exhibited common human clinical symptoms of PDAC such as ascites, jaundice, and bowel obstruction at morbidity at roughly the same frequencies (FIG. 7C). Histopathological analysis revealed that p53 deficiency or expression of either p53 mutant consistently led to the development of adenocarcinomas, with 68-78% of tumors manifesting a moderately/well-differentiated primary tumor grade (FIG. 7D). These tumors were characterized by CK19 positivity and frequently had tumor-adjacent PanINs (FIG. 7E). Histological analysis of livers, the most common site of metastasis, revealed similar rates of metastasis across cohorts (FIGS. 7D & 7F). Each cohort also had evidence of rare metastases to the peritoneum, diaphragm and lung observed upon dissection. Lineage tracing using the tdTomato allele confirmed that all tumors and metastases originated from Ptf1a-expressing cells, suggesting an acinar cell origin (FIGS. 7E-7F).

That mutant p53 expression did not obviously change metastatic rates suggests that there is no clear gain-of-function effect of p53 point mutant expression in enhancing metastasis. Accordingly, ˜25% of KT;Ptf1a^{CreER};Trp53^{fl/LSL-R172H} and KT;Ptf1a^{CreER};Trp53^{fl/LSL-R270H} mice had at least one primary tumor negative for mutant p53 expression, often accompanied by a metastasis negative for p53 expression (FIG. 7G). These observations suggest that there is not an absolute selection for cells expressing mutant p53.

Conclusion: Collectively, these findings demonstrate that, like p53 deficiency, mutant p53 promotes PDAC development, but does not exhibit a clear gain-of-function effect in terms of tumor latency and rates of metastasis in acinar cell-derived pancreatic cancer development.

### Example 3: Trp53 Mutation is Required for PDAC Development from Adult Pancreatic Ductal Cells

Background: Next, it was sought to define the genetic requirements for PDAC development from adult pancreatic ductal cells.

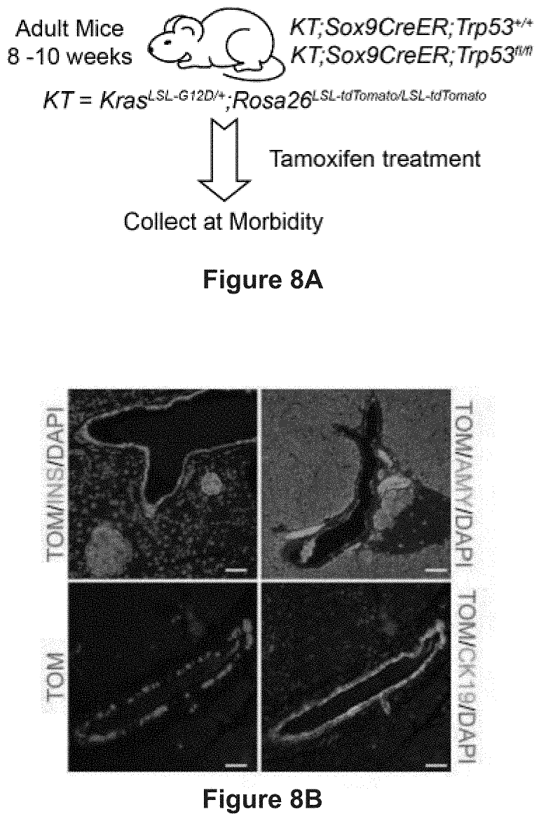

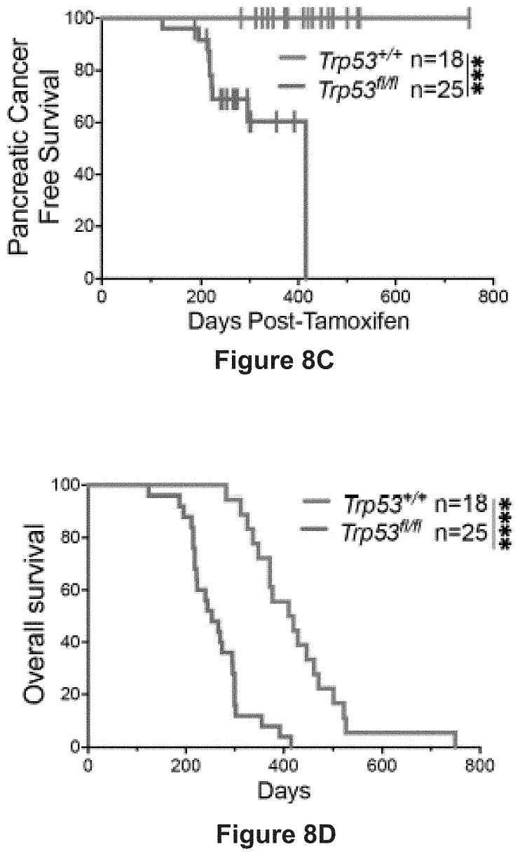

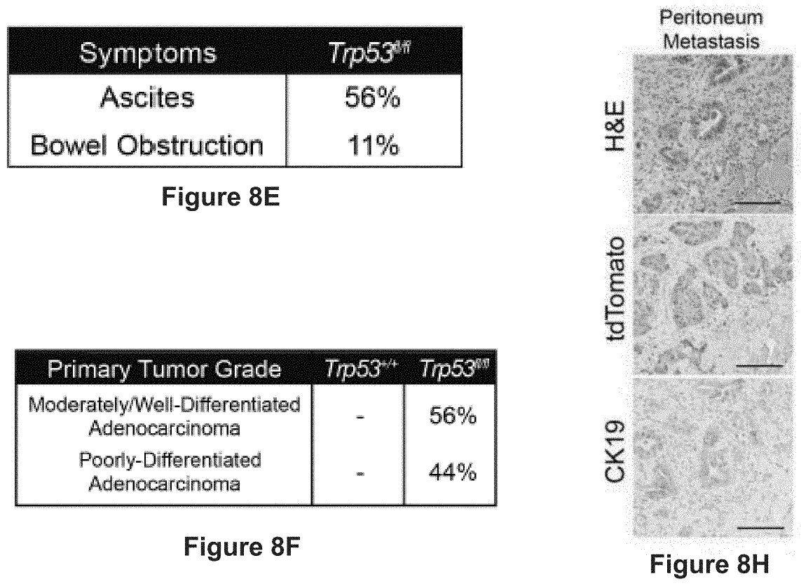

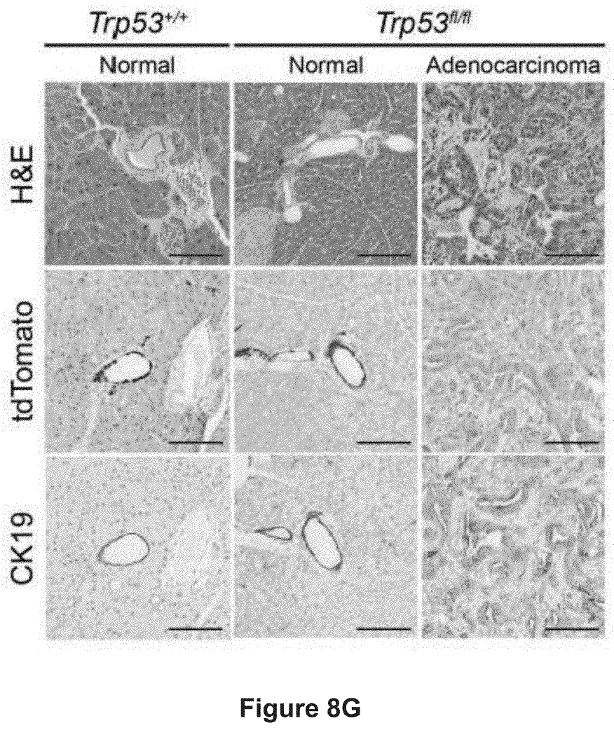

Methods: Toward this end, mouse cohorts were generated in which oncogenic Kras^{G12D} were activated and Trp53 was modulated using the tamoxifen-inducible Sox9CreER transgene (FIG. 8A). Tamoxifen treatment of adult mice resulted in efficient genomic recombination in ductal cells, as indicated by expression of the tdTomato reporter allele in CK19-positive ductal cells (FIG. 8B). First tumor development was assessed in mice expressing or lacking p53 by generating, tamoxifen treating, aging, and examining cohorts of KT;Sox9CreER;Trp53^{fl/fl} and KT;Sox9CreER;Trp53^{+/+}, mice for pancreatic cancer-free survival upon morbidity.

Results: Kaplan Meier analysis revealed that 32% of KT;Sox9CreER;Trp53^{fl/fl} mice developed pancreatic cancer by −300 days post-tamoxifen treatment (FIG. 8C). In contrast, none of the 18 KT;Sox9CreER;Trp53^{+/+} mice developed pancreatic cancer, suggesting that Trp53 loss is necessary for ductal cell-derived pancreatic cancer development. However, the ductal model is complicated by the expression of Sox9 in adult tissues beyond the pancreas, leading to the development of non-pancreatic tumors, such as Harderian gland adenocarcinomas, that necessitates early sacrifice with a median overall survival of 415 days in KT;Sox9CreER;Trp53^{+/+} mice and 253 days in KT;Sox9CreER;Trp53^{fl/fl} mice (FIG. 8D). Pancreata were therefore analyzed when mice reached morbidity due either to pancreatic or non-pancreatic tumors. At morbidity, some KT;Sox9CreER;Trp53^{fl/fl} mice succumbing to PDAC exhibited common human clinical symptoms of PDAC such as ascites and bowel obstruction (FIG. 8E). Both moderately/well-differentiated and poorly-differentiated adenocarcinomas in pancreatic tumor-bearing KT;Sox9CreER;Trp53^{fl/fl} mice were observed at similar frequencies to KT;Ptf1a^{CreER};Trp53^{fl/fl} mice (44% versus 39% poorly-differentiated tumors, respectively; FIGS. 6E & 8F). The adenocarcinomas were characterized by CK19-positivity and importantly, lineage tracing with tdTomato suggested a Sox9-expressing pancreatic cell-of-origin for these tumors (FIG. 8G). Histological analysis and alcian blue staining failed to reveal PanINs associated with tumors in KT;Sox9CreER;Trp53^{fl/fl} mice (FIG. 8G). In terms of establishing the incidence of metastasis, neither tumor histology nor tdTomato positivity could be used to distinguish PDAC metastases from primary cholangiocarcinomas resulting from Sox9CreER activity in the liver where it can promote neoplasia. However, visual inspection upon dissection, coupled with histological analysis, identified small clusters of tdTomato-positive malignant cells in the peritoneum and lungs of mice with clear PDACs, suggesting that metastasis had occurred (FIG. 8H).

Next, to assess the gain-of-function phenotypes of mutant p53 expression in adult ductal cell-derived pancreatic cancer development, mouse cohorts were generated with oncogenic KRAS^{G12D} expression and expression of the structural mutant p53^{R172H}(KT;Sox9CreER;Trp53^{fl/LSL-R172H}), expression of the contact mutant p53^{R270H} (KT;Sox9CreER;Trp53^{fl/LSL-R270H}), or conditional Trp53 knockout (KT;Sox9CreER;Trp53^{fl/-}). As with the acinar model, these mice were generated to create uniform Trp53 heterozygous stroma. Mice in all three cohorts displayed similar pancreatic cancer-free and overall survivals and exhibited clinical symptoms of pancreatic cancer development. However, it was unclear if there was a gain-of-function phenotype as the incidence of pancreatic cancer development was low. Histopathological analysis revealed the development of poorly to moderately/well-differentiated adenocarcinomas in all cohorts and a sarcomatoid carcinoma in one KT;Sox9CreER;Trp53^{fl/-} mouse. Again, the adenocarcinomas were characterized by CK19 expression and tdTomato expression, confirming a Sox9-expressing pancreatic cell origin for these tumors.

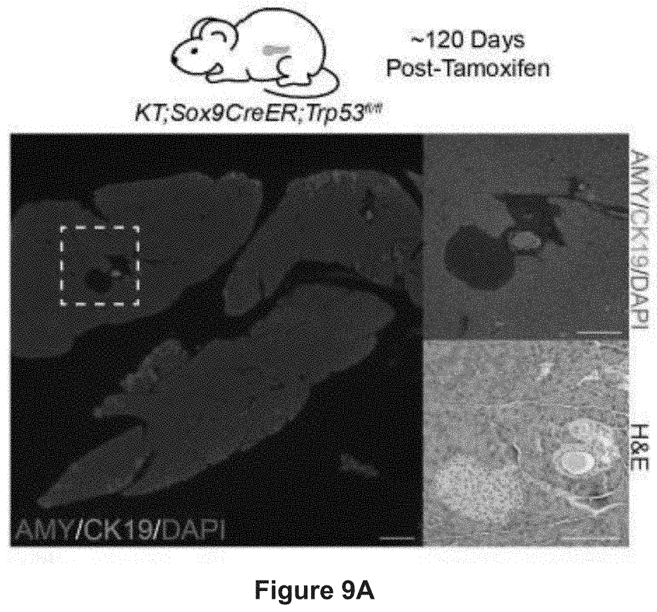

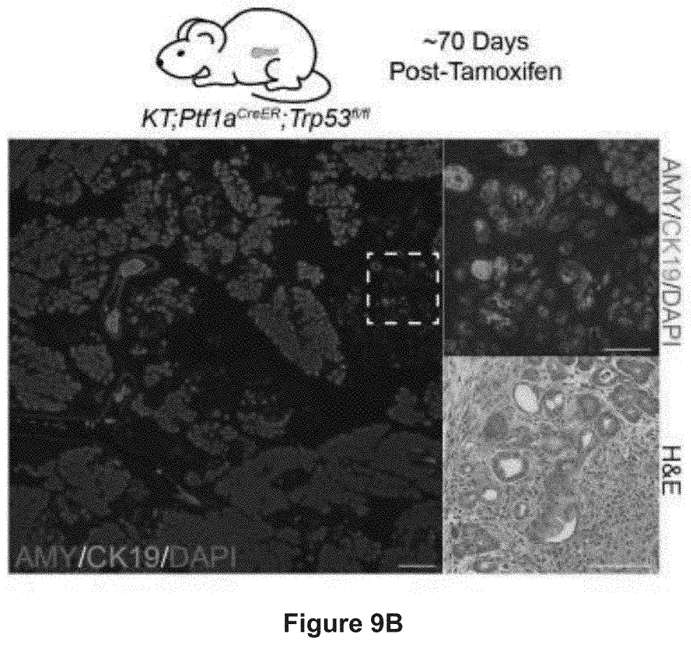

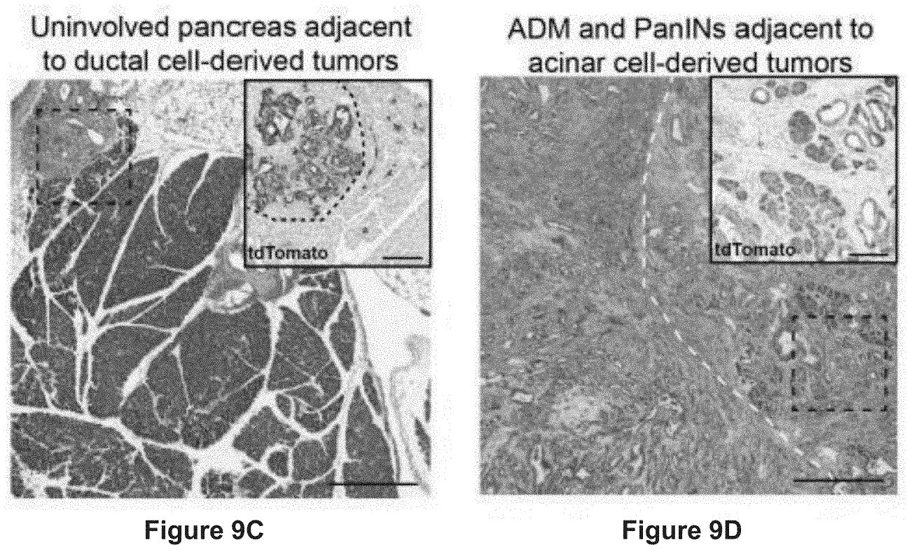

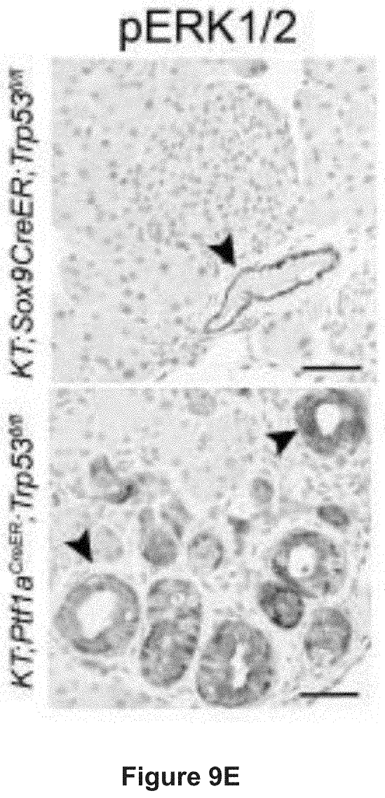

To investigate a potential precursor lesion for the ductal cell-derived tumors, the pancreata of mice were examined prior to cancer development. Analysis of KT;Sox9CreER;Trp53^{fl/fl} mice at −120 days—the midpoint of median overall survival in the ductal model—consistently failed to reveal evidence of potential precursor lesions, including ADMs or PanINs (FIG. 9A). In stark contrast, KT;Ptf1a^{CreER};Trp53^{fl/fl} mice showed early evidence of ADM and PanIN development at −70 days, the midpoint of median overall survival in the acinar model (FIG. 9B). When ductal cell-derived tumors developed, tumors were often were surrounded by uninvolved pancreas (FIG. 9C), whereas acinar cell-derived tumors were typically surrounded by ADMs and PanINs (FIG. 9D). Notably, some ducts in the pancreata of KT;Sox9CreER;Trp53^{fl/fl} mice displayed phospho-ERK1/2 staining, as did ADMs and PanINs of KT;Ptf1a^{CreER};Trp53^{fl/fl} mice, indicative of KRAS activation in these cells (FIG. 9E). This observation, coupled with the lack other precursor lesions, suggests that ductal cell-derived tumors might originate directly from ducts.

Conclusion: Collectively, these findings demonstrate that oncogenic KRAS expression and Trp53 inactivation or mutation can drive pancreatic cancer development directly from adult mouse ductal cells, highlighting a role for p53 in blocking pancreatic cancer development from ductal cells in adult mice.

### Example 4: Acinar and Ductal Cell-Derived Tumors are Transcriptionally Distinct

Background: Pancreatic cancer can originate from either PTF1A- or SOX9-expressing pancreatic cells, which may represent acinar and ductal cells, respectively.

Methods: To gain insight into whether the cell-of-origin affects the molecular subtype of PDAC, transcriptomic analysis of bulk acinar cell-derived and ductal cell-derived tumors was performed by RNA-sequencing (RNA-seq).

Results: Tumors with Trp53 deletion were utilized, rather than Trp53 point mutation, given the robust phenotypes observed with p53 deficiency in both tumor models. Notably, the set of acinar cell-derived and ductal-cell derived tumors that were analyzed had similar differentiation profiles. PCA analysis of transcriptomes revealed that acinar cell-derived and ductal cell-derived tumors cluster distinctly, suggestive of different molecular profiles (FIG. 1A). Indeed, analysis of differentially-expressed genes by DESeq2 revealed that 1,075 genes are more highly expressed in acinar cell-derived tumors than in ductal cell-derived tumors and that 417 genes are more highly expressed in ductal cell-derived tumors than in acinar cell-derived tumors (log₂ fold change>1.0, p-adjusted value<0.05; FIG. 1B). Immunohistochemical analysis confirmed the differential expression of proteins encoded by several of these genes, including CLDN18, TFF1, and MUC5AC, all of which showed higher expression in acinar cell-derived tumors than in ductal cell-derived tumors (FIG. 1C).

Comparison of the acinar and ductal cell-derived tumor signatures to single cell RNA-seq data for normal pancreatic acinar and ductal cells failed to reveal a significant likeness of tumors to the cell-of-origin (FIG. 1D). Functional annotation revealed pathways specific to tumors derived from each cell-of-origin (FIGS. 1E-1F). Gene Ontology analysis of the top pathways enriched in acinar cell-derived tumors revealed categories linked to extracellular matrix (ECM) organization, cell adhesion and digestive system development (FIG. 1E). Consistent with the enhanced ECM organization, Masson's Trichrome staining revealed increased collagen staining in acinar cell-derived tumors compared to ductal cell-derived tumors (FIG. 1G). In contrast to the acinar cell-derived tumors, the ductal cell derived-tumors displayed enrichment for GO terms such as ribosome function and glycolysis (FIG. 1F).

Conclusion: These findings demonstrate that the molecular profile of pancreatic tumors is dependent on the cell-of-origin.

### Example 5: Acinar and Ductal Cell-Derived Tumor Signatures Correlate with Distinct Molecular Subtypes of Human PDAC

Background: As described in Examples 1-4, human PDAC has been classified into discrete molecular subtypes by transcriptomic analyses. It was hypothesized that the emergence of these tumor subtypes could be influenced by cell-of-origin. It was therefore sought to determine whether the acinar cell-derived and ductal cell-derived tumor signatures are enriched in different molecular subtypes of human pancreatic cancer.

Methods: Acinar cell-derived and ductal cell-derived tumor signatures were defined based on the highest-confidence differentially-expressed mouse genes, using a log₂ fold change>1.5 and p-adjusted value<0.01. We thus identified 640 genes as differentially-expressed between acinar cell-derived and ductal cell-derived tumors, 573 of which had human orthologs (FIG. 2A). Of these, 496 genes were more highly expressed in acinar cell-derived tumors than in ductal cell-derived tumors and 77 genes were more highly expressed in ductal cell-derived tumors than in acinar cell-derived tumors.

Results: To investigate whether these defined signatures were associated with particular human PDAC subtypes, we interrogated whether there is an enrichment of either the acinar cell-derived tumor signature or ductal cell-derived tumor signature in human PDACs classified using published datasets. We began with the 4 molecular subtype classification scheme—squamous, pancreatic progenitor, immunogenic, and abnormally differentiated endocrine exocrine (ADEX)—defined through analysis of the Australian International Cancer Genome Initiative (ICGC) patient cohort using RNA-seq of untreated bulk primary PDACs of high cellularity. Notably, generation of this data set through RNA-seq analysis of bulk primary tumors mirrors the input material and methodology of our mouse gene expression analysis. Strikingly, gene set variation analysis (GSVA) of these data revealed a significant enrichment of the ductal cell-derived tumor signature in tumors of the squamous subtype (FIGS. 2F-2H). In contrast, the acinar cell-derived tumor signature was significantly enriched in the pancreatic progenitor, ADEX, and immunogenic subtypes—which are now thought to be within a single broad subtype known as classical—relative to the squamous subtype (FIGS. 2F & 2H). To confirm subtype enrichment of the cell-of-origin signatures in an independent cohort, we leveraged an RNA-seq data set of bulk primary PDACs from The Cancer Genome Atlas (TCGA) Research Network, classified according to the four subtype classification scheme. Again, GSVA uncovered a significant enrichment of the ductal cell-derived tumor signature in the squamous subtype relative to the pancreatic progenitor, immunogenic and ADEX subtypes. In contrast, the acinar cell-derived tumor signature was significantly enriched in the pancreatic progenitor, ADEX, and immunogenic subtypes relative to the squamous subtype.

The patterns of enrichment of signatures were then compared in the Australian ICGC PDAC patient cohort processed using the other major classification schemes. The ICGC data set classified using the classical, quasi-mesenchymal, and exocrine-like molecular subtype designations, which were derived from microarray analysis of microdissected epithelium of untreated, primary PDACs, revealed a significant enrichment of the ductal cell-derived tumor signature in tumors classified as the quasi-mesenchymal subtype relative to the other subtypes (FIGS. 2I-2J). In contrast, the acinar cell-derived tumor signature was significantly enriched in the classical and exocrine-like subtypes relative to the quasi-mesenchymal subtype (FIGS. 2I & 2K). The Australian ICGC PDAC patient data was then classified using the basal-like and classical designations originally derived from analysis of bulk, resected untreated primary PDACs and metastases by both microarrays and RNA-seq, followed by the exclusion of transcripts expressed in the normal pancreas. The ductal cell-derived tumor signature was significantly enriched in basal-like tumors (FIGS. 2L-2M) and the acinar cell-derived tumor signature was significantly enriched in classical tumors (FIGS. 2L & 2N). Subsequent studies have led to the consensus that there are two broad subtypes—the basal-like (including the squamous or quasi-mesenchymal subtypes) and classical subtype (including the pancreatic progenitor, immunogenic, ADEX, and exocrine-like subtypes).

Conclusion: These findings suggest that the acinar cell-derived tumor signature is associated with the classical subtype and the ductal cell-derived tumor signature correlates with the basal-like subtype.

### Example 6: The Cell-of-Origin Signatures are Associated with Patient Survival Outcomes

Background: Next the clinical significance of cell-of-origin signatures was evaluated by examining associations with PDAC patient overall survival in the Australian ICGC cohort.

Results: Interestingly, patients whose tumors showed low expression of the acinar cell-derived signature had significantly worse overall survival than those with high expression of the acinar signature (FIG. 3A). In contrast, patients whose tumors showed high expression of the ductal cell-derived signature had significantly worse overall survival than those with low expression of the ductal signature (FIG. 3B). Cox proportional hazard analysis demonstrated that high expression of the ductal cell-derived signature remained significantly associated with decreased overall survival in a model that incorporated key clinical covariates such as age, tumor stage and grade and subtype classification, while high expression of the acinar cell derived signature was independently associated with improved overall survival (FIG. 3C). These independent survival associations were confirmed in a meta-analysis of three PDAC patient cohorts, including the Australian and Canadian ICGC cohorts and the TCGA cohort (FIG. 3D). Similar to the Australian ICGC cohort, the Canadian ICGC cohort again demonstrated significant associations of the acinar cell-derived signature with improved survival and of the ductal cell-derived signature with decreased survival. It is unclear why the TCGA cohort alone does not reflect the same outcome associations as the ICGC cohorts, but this could be related to differences in epithelial purity of these samples.

Conclusion: Collectively, these findings indicate that the acinar and ductal cell-derived tumor signatures are independently associated with survival of human PDAC patients. Moreover, these findings demonstrate that the very earliest events in carcinogenesis can impart on tumors a fundamental program that drives cancer subtype and phenotype.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

| hg.ensemble.gene.id | hgnc.symbol | Tumor Signature | hg.ensemble.gene.id | hgnc.symbol | Tumor Signature |
|---|---|---|---|---|---|
| ENSG00000121068 | TBX2 | Acinar cell-derived | ENSG00000141574 | SECTM1 | Acinar cell-derived |
| ENSG00000118971 | CCND2 | Acinar cell-derived | ENSG00000119514 | GALNT12 | Acinar cell-derived |
| ENSG00000285901 | AC008012.1 | Acinar cell-derived | ENSG00000128266 | GNAZ | Acinar cell-derived |
| ENSG00000205045 | SLFN12L | Acinar cell-derived | ENSG00000050628 | PTGER3 | Acinar cell-derived |
| ENSG00000172123 | SLFN12 | Acinar cell-derived | ENSG00000146122 | DAAM2 | Acinar cell-derived |
| ENSG00000254647 | INS | Acinar cell-derived | ENSG00000162645 | GBP2 | Acinar cell-derived |
| ENSG00000115263 | GCG | Acinar cell-derived | ENSG00000116299 | KIAA1324 | Acinar cell-derived |
| ENSG00000077063 | CTTNBP2 | Acinar cell-derived | ENSG00000090006 | LTBP4 | Acinar cell-derived |
| ENSG00000119866 | BCL11A | Acinar cell-derived | ENSG00000138075 | ABCG5 | Acinar cell-derived |
| ENSG00000007216 | SLC13A2 | Acinar cell-derived | ENSG00000007312 | CD79B | Acinar cell-derived |
| ENSG00000091138 | SLC26A3 | Acinar cell-derived | ENSG00000189337 | KAZN | Acinar cell-derived |
| ENSG00000130176 | CNN1 | Acinar cell-derived | ENSG00000120278 | PLEKHG1 | Acinar cell-derived |
| ENSG00000019102 | VSIG2 | Acinar cell-derived | ENSG00000157554 | ERG | Acinar cell-derived |
| ENSG00000156299 | TIAM1 | Acinar cell-derived | ENSG00000169891 | REPS2 | Acinar cell-derived |
| ENSG00000164690 | SHH | Acinar cell-derived | ENSG00000166265 | CYYR1 | Acinar cell-derived |
| ENSG00000105369 | CD79A | Acinar cell-derived | ENSG00000001626 | CFTR | Acinar cell-derived |
| ENSG00000241644 | INMT | Acinar cell-derived | ENSG00000173269 | MMRN2 | Acinar cell-derived |
| ENSG00000254959 | INMT-MINDY4 | Acinar cell-derived | ENSG00000204176 | SYT15 | Acinar cell-derived |
| ENSG00000157005 | SST | Acinar cell-derived | ENSG00000277758 | FO681492.1 | Acinar cell-derived |
| ENSG00000127990 | SGCE | Acinar cell-derived | ENSG00000069188 | SDK2 | Acinar cell-derived |
| ENSG00000242114 | MTFP1 | Acinar cell-derived | ENSG00000100344 | PNPLA3 | Acinar cell-derived |
| ENSG00000072952 | MRVI1 | Acinar cell-derived | ENSG00000079841 | RIMS1 | Acinar cell-derived |
| ENSG00000157404 | KIT | Acinar cell-derived | ENSG00000121351 | IAPP | Acinar cell-derived |
| ENSG00000099994 | SUSD2 | Acinar cell-derived | ENSG00000172572 | PDE3A | Acinar cell-derived |
| ENSG00000077942 | FBLN1 | Acinar cell-derived | ENSG00000154258 | ABCA9 | Acinar cell-derived |
| ENSG00000120156 | TEK | Acinar cell-derived | ENSG00000136237 | RAPGEF5 | Acinar cell-derived |
| ENSG00000123191 | ATP7B | Acinar cell-derived | ENSG00000138615 | CILP | Acinar cell-derived |
| ENSG00000283632 | EXOC3L2 | Acinar cell-derived | ENSG00000174502 | SLC26A9 | Acinar cell-derived |
| ENSG00000244486 | SCARF2 | Acinar cell-derived | ENSG00000189056 | RELN | Acinar cell-derived |
| ENSG00000128918 | ALDH1A2 | Acinar cell-derived | ENSG00000187068 | C3orf70 | Acinar cell-derived |
| ENSG00000196092 | PAX5 | Acinar cell-derived | ENSG00000196542 | SPTSSB | Acinar cell-derived |
| ENSG00000134716 | CYP2J2 | Acinar cell-derived | ENSG00000077616 | NAALAD2 | Acinar cell-derived |
| ENSG00000130234 | ACE2 | Acinar cell-derived | ENSG00000176273 | SLC35G1 | Acinar cell-derived |
| ENSG00000172379 | ARNT2 | Acinar cell-derived | ENSG00000188175 | HEPACAM2 | Acinar cell-derived |
| ENSG00000117594 | HSD11B1 | Acinar cell-derived | ENSG00000137634 | NXPE4 | Acinar cell-derived |
| ENSG00000159307 | SCUBE1 | Acinar cell-derived | ENSG00000134817 | APLNR | Acinar cell-derived |
| ENSG00000137573 | SULF1 | Acinar cell-derived | ENSG00000180730 | SHISA2 | Acinar cell-derived |
| ENSG00000131096 | PYY | Acinar cell-derived | ENSG00000160886 | LY6K | Acinar cell-derived |
| ENSG00000108849 | PPY | Acinar cell-derived | ENSG00000135083 | CCNJL | Acinar cell-derived |
| ENSG00000273171 | AC002094.3 | Acinar cell-derived | ENSG00000146285 | SCML4 | Acinar cell-derived |
| ENSG00000109072 | VTN | Acinar cell-derived | ENSG00000139973 | SYT16 | Acinar cell-derived |
| ENSG00000179761 | PIPOX | Acinar cell-derived | ENSG00000081818 | PCDHB4 | Acinar cell-derived |
| ENSG00000164749 | HNF4G | Acinar cell-derived | ENSG00000174937 | OR5M3 | Acinar cell-derived |
| ENSG00000100979 | PLTP | Acinar cell-derived | ENSG00000185477 | GPRIN3 | Acinar cell-derived |
| ENSG00000161405 | IKZF3 | Acinar cell-derived | ENSG00000181072 | CHRM2 | Acinar cell-derived |
| ENSG00000185811 | IKZF1 | Acinar cell-derived | ENSG00000129422 | MTUS1 | Acinar cell-derived |
| ENSG00000133392 | MYH11 | Acinar cell-derived | ENSG00000176435 | CLEC14A | Acinar cell-derived |
| ENSG00000015413 | DPEP1 | Acinar cell-derived | ENSG00000198133 | TMEM229B | Acinar cell-derived |
| ENSG00000131471 | AOC3 | Acinar cell-derived | ENSG00000120327 | PCDHB14 | Acinar cell-derived |
| ENSG00000181392 | SYNE4 | Acinar cell-derived | ENSG00000169291 | SHE | Acinar cell-derived |
| ENSG00000009765 | IYD | Acinar cell-derived | ENSG00000203883 | SOX18 | Acinar cell-derived |
| ENSG00000112379 | ARFGEF3 | Acinar cell-derived | ENSG00000145861 | C1QTNF2 | Acinar cell-derived |
| ENSG00000172594 | SMPDL3A | Acinar cell-derived | ENSG00000161267 | BDH1 | Acinar cell-derived |
| ENSG00000196569 | LAMA2 | Acinar cell-derived | ENSG00000187908 | DMBT1 | Acinar cell-derived |
| ENSG00000185002 | RFX6 | Acinar cell-derived | ENSG00000149968 | MMP3 | Acinar cell-derived |
| ENSG00000154269 | ENPP3 | Acinar cell-derived | ENSG00000133561 | GIMAP6 | Acinar cell-derived |
| ENSG00000079931 | MOXD1 | Acinar cell-derived | ENSG00000187372 | PCDHB13 | Acinar cell-derived |
| ENSG00000017427 | IGF1 | Acinar cell-derived | ENSG00000233670 | PIRT | Acinar cell-derived |
| ENSG00000108187 | PBLD | Acinar cell-derived | ENSG00000163431 | LMOD1 | Acinar cell-derived |
| ENSG00000166148 | AVPR1A | Acinar cell-derived | ENSG00000138185 | ENTPD1 | Acinar cell-derived |
| ENSG00000127329 | PTPRB | Acinar cell-derived | ENSG00000127152 | BCL11B | Acinar cell-derived |
| ENSG00000145936 | KCNMB1 | Acinar cell-derived | ENSG00000046889 | PREX2 | Acinar cell-derived |
| ENSG00000106070 | GRB10 | Acinar cell-derived | ENSG00000196371 | FUT4 | Acinar cell-derived |
| ENSG00000099866 | MADCAM1 | Acinar cell-derived | ENSG00000174992 | ZG16 | Acinar cell-derived |
| ENSG00000105851 | PIK3CG | Acinar cell-derived | ENSG00000187513 | GJA4 | Acinar cell-derived |
| ENSG00000261371 | PECAM1 | Acinar cell-derived | ENSG00000172403 | SYNPO2 | Acinar cell-derived |
| ENSG00000179841 | AKAP5 | Acinar cell-derived | ENSG00000165633 | VSTM4 | Acinar cell-derived |
| ENSG00000196136 | SERPINA3 | Acinar cell-derived | ENSG00000078589 | P2RY10 | Acinar cell-derived |
| ENSG00000258989 | AL355916.3 | Acinar cell-derived | ENSG00000183801 | OLFML1 | Acinar cell-derived |
| ENSG00000027075 | PRKCH | Acinar cell-derived | ENSG00000174099 | MSRB3 | Acinar cell-derived |
| ENSG00000100604 | CHGA | Acinar cell-derived | ENSG00000173928 | SWSAP1 | Acinar cell-derived |
| ENSG00000071246 | VASH1 | Acinar cell-derived | ENSG00000156466 | GDF6 | Acinar cell-derived |
| ENSG00000182218 | HHIPL1 | Acinar cell-derived | ENSG00000160539 | PLPP7 | Acinar cell-derived |
| ENSG00000078401 | EDN1 | Acinar cell-derived | ENSG00000128815 | WDFY4 | Acinar cell-derived |
| ENSG00000170542 | SERPINB9 | Acinar cell-derived | ENSG00000177732 | SOX12 | Acinar cell-derived |
| ENSG00000130957 | FBP2 | Acinar cell-derived | ENSG00000203950 | RTL8A | Acinar cell-derived |
| ENSG00000145824 | CXCL14 | Acinar cell-derived | ENSG00000212747 | RTL8B | Acinar cell-derived |
| ENSG00000087303 | NID2 | Acinar cell-derived | ENSG00000186265 | BTLA | Acinar cell-derived |
| ENSG00000010319 | SEMA3G | Acinar cell-derived | ENSG00000188959 | C9orf152 | Acinar cell-derived |
| ENSG00000266524 | GDF10 | Acinar cell-derived | ENSG00000105122 | RASAL3 | Acinar cell-derived |
| ENSG00000136574 | GATA4 | Acinar cell-derived | ENSG00000152292 | SH2D6 | Acinar cell-derived |
| ENSG00000102678 | FGF9 | Acinar cell-derived | ENSG00000101425 | BPI | Acinar cell-derived |
| ENSG00000132932 | ATP8A2 | Acinar cell-derived | ENSG00000068078 | FGFR3 | Acinar cell-derived |
| ENSG00000092068 | SLC7A8 | Acinar cell-derived | ENSG00000133574 | GIMAP4 | Acinar cell-derived |
| ENSG00000145014 | TMEM44 | Acinar cell-derived | ENSG00000138722 | MMRN1 | Acinar cell-derived |
| ENSG00000091972 | CD200 | Acinar cell-derived | ENSG00000204632 | HLA-G | Acinar cell-derived |
| ENSG00000091986 | CCDC80 | Acinar cell-derived | ENSG00000234745 | HLA-B | Acinar cell-derived |
| ENSG00000069764 | PLA2G10 | Acinar cell-derived | ENSG00000206503 | HLA-A | Acinar cell-derived |
| ENSG00000144857 | BOC | Acinar cell-derived | ENSG00000204642 | HLA-F | Acinar cell-derived |
| ENSG00000080200 | CRYBG3 | Acinar cell-derived | ENSG00000204525 | HLA-C | Acinar cell-derived |
| ENSG00000144820 | ADGRG7 | Acinar cell-derived | ENSG00000182272 | B4GALNT4 | Acinar cell-derived |
| ENSG00000144852 | NR1I2 | Acinar cell-derived | ENSG00000147257 | GPC3 | Acinar cell-derived |
| ENSG00000173175 | ADCY5 | Acinar cell-derived | ENSG00000171812 | COL8A2 | Acinar cell-derived |
| ENSG00000065485 | PDIA5 | Acinar cell-derived | ENSG00000178184 | PARD6G | Acinar cell-derived |
| ENSG00000145192 | AHSG | Acinar cell-derived | ENSG00000069122 | ADGRF5 | Acinar cell-derived |
| ENSG00000139610 | CELA1 | Acinar cell-derived | ENSG00000155093 | PTPRN2 | Acinar cell-derived |
| ENSG00000198203 | SULT1C2 | Acinar cell-derived | ENSG00000070731 | ST6GALNAC2 | Acinar cell-derived |
| ENSG00000115386 | REGIA | Acinar cell-derived | ENSG00000183044 | ABAT | Acinar cell-derived |
| ENSG00000111371 | SLC38A1 | Acinar cell-derived | ENSG00000197467 | COL13A1 | Acinar cell-derived |
| ENSG00000149131 | SERPING1 | Acinar cell-derived | ENSG00000163017 | ACTG2 | Acinar cell-derived |
| ENSG00000087085 | ACHE | Acinar cell-derived | ENSG00000111666 | CHPT1 | Acinar cell-derived |
| ENSG00000175003 | SLC22A1 | Acinar cell-derived | ENSG00000176485 | PLAAT3 | Acinar cell-derived |
| ENSG00000182568 | SATB1 | Acinar cell-derived | ENSG00000161249 | DMKN | Acinar cell-derived |
| ENSG00000096088 | PGC | Acinar cell-derived | ENSG00000062038 | CDH3 | Acinar cell-derived |
| ENSG00000164530 | PI16 | Acinar cell-derived | ENSG00000107562 | CXCL12 | Acinar cell-derived |
| ENSG00000160181 | TFF2 | Acinar cell-derived | ENSG00000125285 | SOX21 | Acinar cell-derived |
| ENSG00000160179 | ABCG1 | Acinar cell-derived | ENSG00000163145 | C1QTNF7 | Acinar cell-derived |
| ENSG00000160182 | TFF1 | Acinar cell-derived | ENSG00000171004 | HS6ST2 | Acinar cell-derived |
| ENSG00000101605 | MYOM1 | Acinar cell-derived | ENSG00000178718 | RPP25 | Acinar cell-derived |
| ENSG00000013016 | EHD3 | Acinar cell-derived | ENSG00000103723 | AP3B2 | Acinar cell-derived |
| ENSG00000138061 | CYP1B1 | Acinar cell-derived | ENSG00000104833 | TUBB4A | Acinar cell-derived |
| ENSG00000131634 | TMEM204 | Acinar cell-derived | ENSG00000157551 | KCNJ15 | Acinar cell-derived |
| ENSG00000143921 | ABCG8 | Acinar cell-derived | ENSG00000112419 | PHACTR2 | Acinar cell-derived |
| ENSG00000186115 | CYP4F2 | Acinar cell-derived | ENSG00000128052 | KDR | Acinar cell-derived |
| ENSG00000170476 | MZB1 | Acinar cell-derived | ENSG00000106034 | CPED1 | Acinar cell-derived |
| ENSG00000231852 | CYP21A2 | Acinar cell-derived | ENSG00000075035 | WSCD2 | Acinar cell-derived |
| ENSG00000064692 | SNCAIP | Acinar cell-derived | ENSG00000152969 | JAKMIP1 | Acinar cell-derived |
| ENSG00000138829 | FBN2 | Acinar cell-derived | ENSG00000137726 | FXYD6 | Acinar cell-derived |
| ENSG00000156738 | MS4A1 | Acinar cell-derived | ENSG00000132465 | JCHAIN | Acinar cell-derived |
| ENSG00000099139 | PCSK5 | Acinar cell-derived | ENSG00000077274 | CAPN6 | Acinar cell-derived |
| ENSG00000110446 | SLC15A3 | Acinar cell-derived | ENSG00000101335 | MYL9 | Acinar cell-derived |
| ENSG00000133317 | LGALS12 | Acinar cell-derived | ENSG00000143126 | CELSR2 | Acinar cell-derived |
| ENSG00000095596 | CYP26A1 | Acinar cell-derived | ENSG00000134243 | SORT1 | Acinar cell-derived |
| ENSG00000119946 | CNNM1 | Acinar cell-derived | ENSG00000125430 | HS3ST3B1 | Acinar cell-derived |
| ENSG00000166866 | MYO1A | Acinar cell-derived | ENSG00000115598 | IL1RL2 | Acinar cell-derived |
| ENSG00000073417 | PDE8A | Acinar cell-derived | ENSG00000100368 | CSF2RB | Acinar cell-derived |
| ENSG00000163815 | CLEC3B | Acinar cell-derived | ENSG00000164694 | FNDC1 | Acinar cell-derived |
| ENSG00000103942 | HOMER2 | Acinar cell-derived | ENSG00000169397 | RNASE3 | Acinar cell-derived |
| ENSG00000164736 | SOX17 | Acinar cell-derived | ENSG00000169385 | RNASE2 | Acinar cell-derived |
| ENSG00000144407 | PTH2R | Acinar cell-derived | ENSG00000286237 | ARMCX5- | Acinar cell-derived |
| | | | | GPRASP2 | |
| ENSG00000204262 | COL5A2 | Acinar cell-derived | ENSG00000198908 | BHLHB9 | Acinar cell-derived |
| ENSG00000180251 | SLC9A4 | Acinar cell-derived | ENSG00000244731 | C4A | Acinar cell-derived |
| ENSG00000115594 | IL1R1 | Acinar cell-derived | ENSG00000224389 | C4B | Acinar cell-derived |
| ENSG00000115461 | IGFBP5 | Acinar cell-derived | ENSG00000016602 | CLCA4 | Acinar cell-derived |
| ENSG00000175084 | DES | Acinar cell-derived | ENSG00000248771 | SMIM31 | Acinar cell-derived |
| ENSG00000124006 | OBSL1 | Acinar cell-derived | ENSG00000114248 | LRRC31 | Acinar cell-derived |
| ENSG00000079263 | SP140 | Acinar cell-derived | ENSG00000151882 | CCL28 | Acinar cell-derived |
| ENSG00000019169 | MARCO | Acinar cell-derived | ENSG00000019485 | PRDM11 | Acinar cell-derived |
| ENSG00000162896 | PIGR | Acinar cell-derived | ENSG00000204335 | SP5 | Acinar cell-derived |
| ENSG00000163531 | NFASC | Acinar cell-derived | ENSG00000197406 | DIO3 | Acinar cell-derived |
| ENSG00000198734 | F5 | Acinar cell-derived | ENSG00000211599 | IGKV5-2 | Acinar cell-derived |
| ENSG00000106991 | ENG | Acinar cell-derived | ENSG00000241755 | IGKV1-9 | Acinar cell-derived |
| ENSG00000136542 | GALNT5 | Acinar cell-derived | ENSG00000242580 | IGKV1D-43 | Acinar cell-derived |
| ENSG00000162998 | FRZB | Acinar cell-derived | ENSG00000240864 | IGKV1-16 | Acinar cell-derived |
| ENSG00000115232 | ITGA4 | Acinar cell-derived | ENSG00000211633 | IGKV1D-42 | Acinar cell-derived |
| ENSG00000134802 | SLC43A3 | Acinar cell-derived | ENSG00000242766 | IGKV1D-17 | Acinar cell-derived |
| ENSG00000137860 | SLC28A2 | Acinar cell-derived | ENSG00000240671 | IGKV1-8 | Acinar cell-derived |
| ENSG00000134569 | LRP4 | Acinar cell-derived | ENSG00000239855 | IGKV1-6 | Acinar cell-derived |
| ENSG00000089199 | CHGB | Acinar cell-derived | ENSG00000276566 | IGKV1D-13 | Acinar cell-derived |
| ENSG00000125810 | CD93 | Acinar cell-derived | ENSG00000243290 | IGKV1-12 | Acinar cell-derived |
| ENSG00000154930 | ACSS1 | Acinar cell-derived | ENSG00000241244 | IGKV1D-16 | Acinar cell-derived |
| ENSG00000133110 | POSTN | Acinar cell-derived | ENSG00000243466 | IGKV1-5 | Acinar cell-derived |
| ENSG00000090402 | SI | Acinar cell-derived | ENSG00000278857 | IGKV1D-12 | Acinar cell-derived |
| ENSG00000143578 | CREB3L4 | Acinar cell-derived | ENSG00000250036 | IGKV1D-37 | Acinar cell-derived |
| ENSG00000061918 | GUCY1B1 | Acinar cell-derived | ENSG00000242371 | IGKV1-39 | Acinar cell-derived |
| ENSG00000198400 | NTRK1 | Acinar cell-derived | ENSG00000240382 | IGKV1-17 | Acinar cell-derived |
| ENSG00000143387 | CTSK | Acinar cell-derived | ENSG00000239862 | IGKV1-37 | Acinar cell-derived |
| ENSG00000146267 | FAXC | Acinar cell-derived | ENSG00000251546 | IGKV1D-39 | Acinar cell-derived |
| ENSG00000136872 | ALDOB | Acinar cell-derived | ENSG00000239819 | IGKV1D-8 | Acinar cell-derived |
| ENSG00000241697 | TMEFF1 | Acinar cell-derived | ENSG00000211611 | IGKV6-21 | Acinar cell-derived |
| ENSG00000106952 | TNFSF8 | Acinar cell-derived | ENSG00000225523 | IGKV6D-21 | Acinar cell-derived |
| ENSG00000165124 | SVEP1 | Acinar cell-derived | ENSG00000211626 | IGKV6D-41 | Acinar cell-derived |
| ENSG00000122707 | RECK | Acinar cell-derived | ENSG00000211598 | IGKV4-1 | Acinar cell-derived |
| ENSG00000122694 | GLIPR2 | Acinar cell-derived | ENSG00000211592 | IGKC | Acinar cell-derived |
| ENSG00000188921 | HACD4 | Acinar cell-derived | ENSG00000211899 | IGHM | Acinar cell-derived |
| ENSG00000162409 | PRKAA2 | Acinar cell-derived | ENSG00000211951 | IGHV2-26 | Acinar cell-derived |
| ENSG00000158966 | CACHD1 | Acinar cell-derived | ENSG00000211974 | IGHV2-70D | Acinar cell-derived |
| ENSG00000162493 | PDPN | Acinar cell-derived | ENSG00000274576 | IGHV2-70 | Acinar cell-derived |
| ENSG00000117472 | TSPAN1 | Acinar cell-derived | ENSG00000259303 | IGHV2OR16-5 | Acinar cell-derived |
| ENSG00000162551 | ALPL | Acinar cell-derived | ENSG00000211937 | IGHV2-5 | Acinar cell-derived |
| ENSG00000149527 | PLCH2 | Acinar cell-derived | ENSG00000179023 | KLHDC7A | Acinar cell-derived |
| ENSG00000189409 | MMP23B | Acinar cell-derived | ENSG00000095932 | SMIM24 | Acinar cell-derived |
| ENSG00000072201 | LNX1 | Acinar cell-derived | ENSG00000185442 | FAM174B | Acinar cell-derived |
| ENSG00000173597 | SULT1B1 | Acinar cell-derived | ENSG00000112936 | C7 | Acinar cell-derived |
| ENSG00000152583 | SPARCL1 | Acinar cell-derived | ENSG00000186007 | LEMD1 | Acinar cell-derived |
| ENSG00000132938 | MTUS2 | Acinar cell-derived | ENSG00000129048 | ACKR4 | Acinar cell-derived |
| ENSG00000164692 | COL1A2 | Acinar cell-derived | ENSG00000163283 | ALPP | Acinar cell-derived |
| ENSG00000002726 | AOC1 | Acinar cell-derived | ENSG00000163295 | ALPI | Acinar cell-derived |
| ENSG00000163637 | PRICKLE2 | Acinar cell-derived | ENSG00000163286 | ALPG | Acinar cell-derived |
| ENSG00000115353 | TACR1 | Acinar cell-derived | ENSG00000205476 | CCDC85C | Acinar cell-derived |
| ENSG00000283586 | GKN3P | Acinar cell-derived | ENSG00000172752 | COL6A5 | Acinar cell-derived |
| ENSG00000132182 | NUP210 | Acinar cell-derived | ENSG00000280411 | IGHV1-69D | Acinar cell-derived |
| ENSG00000111341 | MGP | Acinar cell-derived | ENSG00000211945 | IGHV1-18 | Acinar cell-derived |
| ENSG00000111348 | ARHGDIB | Acinar cell-derived | ENSG00000211935 | IGHV1-3 | Acinar cell-derived |
| ENSG00000134533 | RERG | Acinar cell-derived | ENSG00000281179 | AC135068.8 | Acinar cell-derived |
| ENSG00000069431 | ABCC9 | Acinar cell-derived | ENSG00000270505 | IGHV1OR15-1 | Acinar cell-derived |
| ENSG00000118308 | LRMP | Acinar cell-derived | ENSG00000188403 | IGHV1OR15-9 | Acinar cell-derived |
| ENSG00000133687 | TMTC1 | Acinar cell-derived | ENSG00000211950 | IGHV1-24 | Acinar cell-derived |
| ENSG00000182253 | SYNM | Acinar cell-derived | ENSG00000211962 | IGHV1-46 | Acinar cell-derived |
| ENSG00000177455 | CD19 | Acinar cell-derived | ENSG00000277282 | IGHV1OR21-1 | Acinar cell-derived |
| ENSG00000137491 | SLCO2B1 | Acinar cell-derived | ENSG00000211973 | IGHV1-69 | Acinar cell-derived |
| ENSG00000133800 | LYVE1 | Acinar cell-derived | ENSG00000281990 | IGHV1-69-2 | Acinar cell-derived |
| ENSG00000121898 | CPXM2 | Acinar cell-derived | ENSG00000211968 | IGHV1-58 | Acinar cell-derived |
| ENSG00000166869 | CHP2 | Acinar cell-derived | ENSG00000278782 | AC136616.3 | Acinar cell-derived |
| ENSG00000005187 | ACSM3 | Acinar cell-derived | ENSG00000211934 | IGHV1-2 | Acinar cell-derived |
| ENSG00000169347 | GP2 | Acinar cell-derived | ENSG00000211961 | IGHV1-45 | Acinar cell-derived |
| ENSG00000078596 | ITM2A | Acinar cell-derived | ENSG00000211938 | IGHV3-7 | Acinar cell-derived |
| ENSG00000102359 | SRPX2 | Acinar cell-derived | ENSG00000211895 | IGHA1 | Acinar cell-derived |
| ENSG00000102362 | SYTL4 | Acinar cell-derived | ENSG00000211890 | IGHA2 | Acinar cell-derived |
| ENSG00000182492 | BGN | Acinar cell-derived | ENSG00000282122 | IGHV7-4-1 | Acinar cell-derived |
| ENSG00000198910 | L1CAM | Acinar cell-derived | ENSG00000211979 | IGHV7-81 | Acinar cell-derived |
| ENSG00000126217 | MCF2L | Acinar cell-derived | ENSG00000174473 | GALNTL6 | Acinar cell-derived |
| ENSG00000104213 | PDGFRL | Acinar cell-derived | ENSG00000197410 | DCHS2 | Acinar cell-derived |
| ENSG00000151617 | EDNRA | Acinar cell-derived | ENSG00000253910 | PCDHGB2 | Acinar cell-derived |
| ENSG00000109511 | ANXA10 | Acinar cell-derived | ENSG00000211679 | IGLC3 | Acinar cell-derived |
| ENSG00000154553 | PDLIM3 | Acinar cell-derived | ENSG00000211685 | IGLC7 | Acinar cell-derived |
| ENSG00000140937 | CDH11 | Acinar cell-derived | ENSG00000254709 | IGLL5 | Acinar cell-derived |
| ENSG00000109436 | TBC1D9 | Acinar cell-derived | ENSG00000211675 | IGLC1 | Acinar cell-derived |
| ENSG00000087245 | MMP2 | Acinar cell-derived | ENSG00000211677 | IGLC2 | Acinar cell-derived |
| ENSG00000205336 | ADGRG1 | Acinar cell-derived | ENSG00000128322 | IGLL1 | Acinar cell-derived |
| ENSG00000285188 | AC008397.2 | Acinar cell-derived | ENSG00000197956 | S100A6 | Ductal cell-derived |
| ENSG00000105650 | PDE4C | Acinar cell-derived | ENSG00000141741 | MIEN1 | Ductal cell-derived |
| ENSG00000179776 | CDH5 | Acinar cell-derived | ENSG00000006625 | GGCT | Ductal cell-derived |
| ENSG00000172831 | CES2 | Acinar cell-derived | ENSG00000281039 | AC005154.5 | Ductal cell-derived |
| ENSG00000176387 | HSD11B2 | Acinar cell-derived | ENSG00000108960 | MMD | Ductal cell-derived |
| ENSG00000110777 | POU2AF1 | Acinar cell-derived | ENSG00000114023 | FAM162A | Ductal cell-derived |
| ENSG00000197580 | BCO2 | Acinar cell-derived | ENSG00000108309 | RUNDC3A | Ductal cell-derived |
| ENSG00000177103 | DSCAML1 | Acinar cell-derived | ENSG00000105974 | CAV1 | Ductal cell-derived |
| ENSG00000118094 | TREH | Acinar cell-derived | ENSG00000150991 | UBC | Ductal cell-derived |
| ENSG00000154133 | ROBO4 | Acinar cell-derived | ENSG00000107485 | GATA3 | Ductal cell-derived |
| ENSG00000076706 | MCAM | Acinar cell-derived | ENSG00000274290 | HIST1H2BE | Ductal cell-derived |
| ENSG00000069974 | RAB27A | Acinar cell-derived | ENSG00000183696 | UPP1 | Ductal cell-derived |
| ENSG00000137809 | ITGA11 | Acinar cell-derived | ENSG00000171848 | RRM2 | Ductal cell-derived |
| ENSG00000166736 | HTR3A | Acinar cell-derived | ENSG00000137203 | TFAP2A | Ductal cell-derived |
| ENSG00000111799 | COL12A1 | Acinar cell-derived | ENSG00000038427 | VCAN | Ductal cell-derived |
| ENSG00000066405 | CLDN18 | Acinar cell-derived | ENSG00000164588 | HCN1 | Ductal cell-derived |
| ENSG00000114812 | VIPR1 | Acinar cell-derived | ENSG00000139890 | REM2 | Ductal cell-derived |
| ENSG00000114737 | CISH | Acinar cell-derived | ENSG00000196876 | SCN8A | Ductal cell-derived |
| ENSG00000111335 | OAS2 | Acinar cell-derived | ENSG00000111669 | TPI1 | Ductal cell-derived |
| ENSG00000101680 | LAMA1 | Acinar cell-derived | ENSG00000175482 | POLD4 | Ductal cell-derived |
| ENSG00000158528 | PPP1R9A | Acinar cell-derived | ENSG00000256514 | AP003419.1 | Ductal cell-derived |
| ENSG00000066056 | TIE1 | Acinar cell-derived | ENSG00000167797 | CDK2AP2 | Ductal cell-derived |
| ENSG00000188641 | DPYD | Acinar cell-derived | ENSG00000175592 | FOSL1 | Ductal cell-derived |
| ENSG00000006740 | ARHGAP44 | Acinar cell-derived | ENSG00000081277 | PKP1 | Ductal cell-derived |
| ENSG00000169604 | ANTXR1 | Acinar cell-derived | ENSG00000118194 | TNNT2 | Ductal cell-derived |
| ENSG00000166106 | ADAMTS15 | Acinar cell-derived | ENSG00000026025 | VIM | Ductal cell-derived |
| ENSG00000164116 | GUCY1A1 | Acinar cell-derived | ENSG00000165698 | SPACA9 | Ductal cell-derived |
| ENSG00000154783 | FGD5 | Acinar cell-derived | ENSG00000125845 | BMP2 | Ductal cell-derived |
| ENSG00000185274 | GALNT17 | Acinar cell-derived | ENSG00000164687 | FABP5 | Ductal cell-derived |
| ENSG00000146205 | ANO7 | Acinar cell-derived | ENSG00000169908 | TM4SF1 | Ductal cell-derived |
| ENSG00000165548 | TMEM63C | Acinar cell-derived | ENSG00000143369 | ECM1 | Ductal cell-derived |
| ENSG00000182107 | TMEM30B | Acinar cell-derived | ENSG00000109743 | BST1 | Ductal cell-derived |
| ENSG00000111110 | PPM1H | Acinar cell-derived | ENSG00000138669 | PRKG2 | Ductal cell-derived |
| ENSG00000138759 | FRAS1 | Acinar cell-derived | ENSG00000159374 | M1AP | Ductal cell-derived |
| ENSG00000136546 | SCN7A | Acinar cell-derived | ENSG00000123095 | BHLHE41 | Ductal cell-derived |
| ENSG00000156219 | ART3 | Acinar cell-derived | ENSG00000152944 | MED21 | Ductal cell-derived |
| ENSG00000154175 | ABI3BP | Acinar cell-derived | ENSG00000010278 | CD9 | Ductal cell-derived |
| ENSG00000160469 | BRSK1 | Acinar cell-derived | ENSG00000157766 | ACAN | Ductal cell-derived |
| ENSG00000080031 | PTPRH | Acinar cell-derived | ENSG00000130477 | UNC13A | Ductal cell-derived |
| ENSG00000129514 | FOXA1 | Acinar cell-derived | ENSG00000102445 | RUBCNL | Ductal cell-derived |
| ENSG00000143891 | GALM | Acinar cell-derived | ENSG00000011376 | LARS2 | Ductal cell-derived |
| ENSG00000107796 | ACTA2 | Acinar cell-derived | ENSG00000102230 | PCYT1B | Ductal cell-derived |
| ENSG00000140835 | CHST4 | Acinar cell-derived | ENSG00000082482 | KCNK2 | Ductal cell-derived |
| ENSG00000165359 | INTS6L | Acinar cell-derived | ENSG00000149646 | CNBD2 | Ductal cell-derived |
| ENSG00000066230 | SLC9A3 | Acinar cell-derived | ENSG00000163584 | RPL22L1 | Ductal cell-derived |
| ENSG00000184307 | ZDHHC23 | Acinar cell-derived | ENSG00000172183 | ISG20 | Ductal cell-derived |
| ENSG00000174944 | P2RY14 | Acinar cell-derived | ENSG00000143507 | DUSP10 | Ductal cell-derived |
| ENSG00000157890 | MEGF11 | Acinar cell-derived | ENSG00000105479 | CCDC114 | Ductal cell-derived |
| ENSG00000087116 | ADAMTS2 | Acinar cell-derived | ENSG00000197747 | S100A10 | Ductal cell-derived |
| ENSG00000109625 | CPZ | Acinar cell-derived | ENSG00000189334 | S100A14 | Ductal cell-derived |
| ENSG00000188747 | NOXA1 | Acinar cell-derived | ENSG00000157064 | NMNAT2 | Ductal cell-derived |
| ENSG00000088280 | ASAP3 | Acinar cell-derived | ENSG00000133067 | LGR6 | Ductal cell-derived |
| ENSG00000150893 | FREM2 | Acinar cell-derived | ENSG00000169083 | AR | Ductal cell-derived |
| ENSG00000163083 | INHBB | Acinar cell-derived | ENSG00000213859 | KCTD11 | Ductal cell-derived |
| ENSG00000162722 | TRIM58 | Acinar cell-derived | ENSG00000089220 | PEBP1 | Ductal cell-derived |
| ENSG00000134323 | MYCN | Acinar cell-derived | ENSG00000165023 | DIRAS2 | Ductal cell-derived |
| ENSG00000136999 | CCN3 | Acinar cell-derived | ENSG00000131981 | LGALS3 | Ductal cell-derived |
| ENSG00000090339 | ICAM1 | Acinar cell-derived | ENSG00000168298 | HIST1H1E | Ductal cell-derived |
| ENSG00000112299 | VNN1 | Acinar cell-derived | ENSG00000128040 | SPINK2 | Ductal cell-derived |
| ENSG00000088899 | LZTS3 | Acinar cell-derived | ENSG00000205076 | LGALS7 | Ductal cell-derived |
| ENSG00000064042 | LIMCH1 | Acinar cell-derived | ENSG00000178934 | LGALS7B | Ductal cell-derived |
| ENSG00000165449 | SLC16A9 | Acinar cell-derived | ENSG00000087128 | TMPRSS11E | Ductal cell-derived |
| ENSG00000132821 | VSTM2L | Acinar cell-derived | ENSG00000176907 | TCIM | Ductal cell-derived |
| ENSG00000215182 | MUC5AC | Acinar cell-derived | ENSG00000172179 | PRL | Ductal cell-derived |
| ENSG00000173320 | STOX2 | Acinar cell-derived | ENSG00000124466 | LYPD3 | Ductal cell-derived |
| ENSG00000007944 | MYLIP | Acinar cell-derived | ENSG00000088726 | TMEM40 | Ductal cell-derived |
| ENSG00000110328 | GALNT18 | Acinar cell-derived | ENSG00000108515 | ENO3 | Ductal cell-derived |
| ENSG00000105929 | ATP6V0A4 | Acinar cell-derived | ENSG00000173404 | INSM1 | Ductal cell-derived |
| ENSG00000174885 | NLRP6 | Acinar cell-derived | ENSG00000100097 | LGALS1 | Ductal cell-derived |
| ENSG00000136457 | CHAD | Acinar cell-derived | ENSG00000115325 | DOK1 | Ductal cell-derived |
| ENSG00000144668 | ITGA9 | Acinar cell-derived | ENSG00000153832 | FBXO36 | Ductal cell-derived |
| ENSG00000141579 | ZNF750 | Acinar cell-derived | ENSG00000142541 | RPL13A | Ductal cell-derived |
| ENSG00000187135 | VSTM2B | Acinar cell-derived | ENSG00000181449 | SOX2 | Ductal cell-derived |
| ENSG00000281887 | GIMAP1- | Acinar cell-derived | ENSG00000197046 | SIGLEC15 | Ductal cell-derived |
| | GIMAP5 | | | | |
| ENSG00000196329 | GIMAP5 | Acinar cell-derived | ENSG00000034510 | TMSB10 | Ductal cell-derived |
| ENSG00000131378 | RFTN1 | Acinar cell-derived | ENSG00000277209 | RPPH1 | Ductal cell-derived |
| | | | ENSG00000261873 | SMIM36 | Ductal cell-derived |

What is claimed is:

1. A method for identifying pancreatic ductal adenocarcinoma (PDAC) subtype comprising:
obtaining or having obtained transcriptomic data derived from a tumor from an individual affected with PDAC; and
identifying a cell origin of the tumor based on the transcriptomic data.

2. The method of claim 1, wherein the cell origin is selected from an acinar cell-derived tumor and a ductal cell-derived tumor.

3. The method of claim 1, further comprising
obtaining a tumor sample from the individual affected with PDAC; and
performing a transcriptomic analysis on the tumor sample.

4. The method of claim 3, wherein the transcriptomic analysis is selected from bulk RNA analysis, spatial transcriptomic analysis, and single cell RNA sequencing.

5. The method of claim 3, wherein the transcriptomic analysis uses a microarray.

6. The method of claim 3, further comprising determining a treatment response for the tumor.

7. The method of claim 6, wherein determining a treatment response comprises:
culturing a cell isolated from the tumor;
providing a treatment to the cell culture;
incubating the cell culture; and
determining a response to the treatment based on a cell viability in the cell culture cells after incubation.

8. The method of claim 7, further comprising treating the individual affected with PDAC based on the response to the treatment.

9. The method of claim 1, further comprising treating the individual affected with PDAC based on whether the tumor is an acinar cell-derived tumor or a ductal cell-derived tumor.

10. The method of claim 9, wherein the tumor is a ductal cell-derived tumor.

11. The method of claim 10, wherein treating the individual affected with PDAC comprises targeting the glycolysis pathway.

12. The method of claim 10, wherein treating the individual affected with PDAC comprises administering at least one of TDZD-8 and 2-DG to the individual affected with PDAC.

13. The method of claim 9, wherein the tumor is an acinar cell-derived tumor.

14. The method of claim 13, wherein treating the individual affected with PDAC comprises inhibiting AKT kinase.

15. The method of claim 13, wherein treating the individual affected with PDAC comprises administering Capivasertib to the individual affected with PDAC.

16. A method for determining treatment response for a pancreatic ductal adenocarcinoma (PDAC) tumor comprising:
obtaining a culture of cancer cells;
providing a treatment to the culture of cancer cells;
incubating the culture of cancer cells; and
determining a response to the treatment based on a cell viability in the culture of cancer cells after incubation.

17. The method of claim 16, the cells in the culture of cancer cells are derived from PDAC tumor.

18. The method of claim 17, wherein the cells in the culture of cancer cells are derived from an acinar cell-derived tumor or a ductal cell-derived tumor.

19. The method of claim 16, wherein determining a response to the treatment comprises performing a cell viability assay selected from manual cell counting, flow cytometry, or performing a methyltransferase assay.

20. The method of claim 16, further comprising transcriptionally profiling the culture of cancer cells to identify the cell of origin of the cells.
